(19) United States
(12) Patent Application Publication
(10) Pub. No.: US 2024/0358830 A1
(43) Pub. Date: Oct. 31, 2024

(54) ANTIGEN PRESENTING CELL/TARGET CELL HYBRIDOMA-DERIVED VACCINES

(71) Applicant: THE REGENTS OF THE UNIVERSITY OF CALIFORNIA, Oakland, CA (US)

(72) Inventors: Young Jik Kwon, Irvine, CA (US); Jee Young Chung, Irvine, CA (US)

(21) Appl. No.: 18/684,155

(22) PCT Filed: Aug. 19, 2022
(86) PCT No.: PCT/US2022/040963

§ 371 Date: Feb. 15, 2024

Related U.S. Application Data
(60) Provisional application No. 63/235,100, filed on Aug. 19, 2021.

(51) Int. Cl. A61K 39/00; A61K 35/15; A61K 39/39; A61P 35/00; C12N 5/16

(52) U.S. Cl. CPC A61K 39/4622; A61K 35/15; A61K 39/39; A61K 39/4615; A61K 39/4644; A61P 35/00; C12N 5/163

(57) ABSTRACT

The disclosure provides compositions, preparations, and methods comprising cell-derived vesicles induced using a blebbing agent from a hybridoma of a dendritic cell and a target cell, and uses thereof, including as a means to modulate a subject's immune system.

### CROSS REFERENCE TO RELATED APPLICATIONS

This application claims priority under 35 U.S.C. § 119 from Provisional Application Ser. No. 63/235,100 filed Aug. 19, 2021, the disclosure of which is incorporated herein by reference.

### TECHNICAL FIELD

The disclosure provides compositions, preparations and methods comprising extracellular blebs produced from a hybridoma comprising an antigen presenting cell and a target cell, and uses thereof, including to modulate a subject's immune system.

### BACKGROUND

Dendritic cells (DCs) are potent antigen presenting cells (APCs) that have been used extensively for the development of vaccines and immunotherapies. DCs have historically proven to be safe in clinical trials and can be engineered from allogenic sources or from the patient's own cells for personalized medicine. Despite proven immunogenicity and favorable safety profiles, DC-based immunotherapies have not achieved significant objective clinical responses.

### SUMMARY

The disclosure provides compositions, preparations (e.g., vaccine preparations), and methods comprising extracellular blebs (EBs) produced from a hybridoma comprising an antigen presenting cell and a target cell (e.g., a cancer cell). The preparations and compositions comprising the EBs disclosed herein are cell-like, but cell-free. The EBs are produced from a hybridoma disclosed herein via a chemically or physically induced blebbing process which is rapid, efficient, and cost- and labor-friendly. As shown in the studies presented herein, EBs produced from hybridomas of dendritic cell and cancer cells were shown to present antigenic proteins in complex with MHC class I and MHC class II molecules to activate T cells with fully optimized molecular machineries in vivo.

In a particular embodiment, the disclosure provides a vaccine preparation comprising extracellular blebs from a hybridoma of an antigen presenting cell and a target cell, wherein the hybridoma expresses an antigen that can modulate a subject's immune system, wherein the extracellular blebs are produced from the hybridoma by treating the hybridoma with a blebbing agent, and wherein the antigen is displayed on the surface of the extracellular blebs. In a further embodiment, the antigen presenting cell is selected from a macrophage, a B cell and a dendritic cell. In yet a further embodiment, the dendritic cell is a mature dendritic cell. In another embodiment, the dendritic cell is an immature dendritic cell. In yet another embodiment, the dendritic cell is a bone marrow derived dendritic cell, a monocyte-derived dendritic cell, or a peripheral blood mononuclear cell-derived dendritic cell. In a further embodiment, the dendritic cell is a human dendritic cell. In yet a further embodiment, the target cell is selected from a cancer cell, an abnormal or diseased cell, a cell engineered to display an antigen, or a cell that is infected with an infectious agent. In a particular embodiment, the target cell is a cancer cell selected from a myeloma, a lymphoma, a carcinoma, a sarcoma, a leukemia, an adenocarcinoma, a thymoma, or a neoplastic cell from a malignant tumor. In another embodiment, the target cell is a cell that is infected by a virus, a fungus, or a bacterium. Examples of viruses include, but are not limited to, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Coronavirus, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, Epstein-Barr virus, European bat lyssavirusalitis, GB virus C/Hepatitis G virus Pegivirus, Hantan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus, Human herpesvirus, Human immunodeficiency virus, Human papillomavirus, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Isfahan virus, JC polyomavirus, Japanese encephalitis virus, Junin arenavirus, KI Polyomavirus, Kunjin virus, Lagos bat virus, Lake Victoria Marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, Semliki forest virus, Seoul virus, Simian foamy virus, Simian virus, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus O, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, and Zika virus. Examples of fungi include, but are not limited to, Absidia corymbifera, Absidia ramose, Achorion gallinae, Actinomadura spp., Ajellomyces dermatididis, Aleurisma brasiliensis, Allersheria boydii, Arthroderma spp., Aspergillus flavus, Aspergillus fumigatu, Basidiobolus spp., Blastomyces spp., Cadophora spp., Candida albicans, Cercospora apii, Chrysosporium spp., Cladosporium spp., Cladothrix asteroids, Coccidioides immitis, Cryptococcus albidus, Cryptococcus gattii, Cryptococcus laurentii, Cryptococcus neoformans, Cunninghamella elegans, Dematium wernecke, Discomyces israelii, Emmonsia spp., Emmonsiella capsulate, Endomyces geotrichum, Entomophthora coronate, Epidermophyton floccosum, Filobasidiella neoformans, Fonsecaea spp., Geotrichum candidum, Glenospora khartoumensis, Gymnoascus gypseus, Haplosporangium parvum, Histoplasma, Histoplasma capsulatum, Hormiscium dermatididis, Hormodendrum spp., Keratinomyces spp., Langeronia soudanense, Leptosphaeria senegalensis, Lichtheimia corymbifera, Lobmyces loboi., Loboa loboi, Lobomycosis, Madurella spp., Malassezia furfur, Micrococcus pelletieri, Microsporum spp., Monilia spp., Mucor spp., Mycobacterium tuberculosis, Nannizzia spp., Neotestudina rosatii, Nocardia spp., Oidium albicans, Oospora lactis, Paracoccidioides brasiliensis, Petriellidium boydii, Phialophora spp., Piedraia hortae, Pityrosporum furfur, Pneumocystis jirovecii (or Pneumocystis carinii), Pullularia gougerotii, Pyrenochaeta romeroi, Rhinosporidium seeberi, Sabouraudites (Microsporum), Sartorya fumigate, Sepedonium, Sporotrichum spp., Stachybotrys, Stachybotrys chartarum, Streptomyce spp., Tinea spp., Torula spp., Trichophyton spp., Trichosporon spp., and Zopfia rosatii. Examples of bacteria include, but are not limited to, Actinomyces israelii, Bacillus anthracis, Bacillus cereus, Bartonella henselae, Bartonella quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Francisella tularensis, Haemophilus influenzae, Helicobacter pylon, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumoniae, Neisseria gonorrhoeae, Neisseria meningitidis, Pseudomonas aeruginosa, Rickettsia rickettsia, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Ureaplasma urealyticum, Vibrio cholerae, Yersinia pestis, Yersinia enterocolitica, and Yersinia pseudotuberculosis. In another embodiment, the antigen presenting cell and/or target cell is from a subject that is to be treated with the vaccine preparation. In yet another embodiment, the antigen comprises a tumor-specific antigen, or a tumor-associated antigen. Examples of tumor-specific antigens and tumor-associated antigens include, but are not limited to, alphafetoprotein, carcinoembryonic antigen, CA-125, MUC-1, epithelial tumor antigen, tyrosinase, melanoma-associated antigen, k-ras, abnormal products of p53, alpha-actinin-4, ARTC1, B-RAF, BCR-ABL fusion protein, beta-catenin, CASP-5, CASP-8, CDc27, CDK12, CDK4, CDKN2A, CLPP, COA-1, COA-2, CSNK1A1, dek-can fusion protein, EFTUD2, Elongation factor 2, ETV6-AML1 fusion protein, FLT3-ITD, FN1, FNDC3B, GAS7, GPNMB, HAUS3, HLA-A11, HLA-A2, hsp70-2, LDLR-fucosyltransferaseAS fusion protein, MART2, MATN, MEl, MUM-1, MUM-2, MUM-3, Myosin class I, N-ras, neo-PAP, NFYC, OGT, OS-9, p53, pml-RARalpha fusion protein, PPP1R3B, PRDX5, PTPRK, RBAF600, SIRT2, SNRPD1, SYT-SSX1 or -SSX2 fusion protein, TGF-betaRII, TP53, Triosephosphate isomerase, BAGE-1, CT37/FMR1NB, Cyclin-A1, D393-CD20n, GAGE-1,2,8, GAGE-3,4,5,6,7, GnTV, HERV-E, HERV-K-MEL, KK-LC-1, KM-HIN-1, LAGE-1, LRPAP1, LY6K, MAGE-A1, MAGE-A10, MAGE-A12m, MAGE-A2, MAGE-A3, MAGE-A4, MAGE-C1, MAGE-C2, mucin, NA88-A, NY-ESO-1/LAGE-2, SAGE, Sp17, SSX-2, SSX-4, TAG-1, TAG-2, TRAG-3, TRP2-INT2, XAGE-1b/GAGED2a, CEA, gp100/Pmel17, mammaglobin-A, Melan-A/MART-1, NY-BR-1, OA1, PAP, PSA, RAB38/NY-MEL-1, TRP-1/gp75, TRP-2, BCLX, BING-4, CALCA, CD274, CD45, CPSF, cyclin D1, DKK1, ENAH, epCAM, EphA3, EZH2, FGF5, glypican-3, HEPACAM, Hepsin, HER-2/neu, HLA-G, HSPH1, IGF2B3, IMP-3, MUC1, Meloe, Midkine, WTi, VEGF, TPBG, telomerase, STEAPI, STEAPI, RAGE-1, PSMA, PRAME, RGS5, RhoC, RNF43, RU2AS, SOX10, and survivin. In a further embodiment, the antigen is a foreign antigen or a self-antigen. In yet a further embodiment, the foreign antigen is from a bacterium, fungus or virus. In another embodiment, the vaccine preparation further comprises an adjuvant. Examples of adjuvants include, but are not limited to, aluminum hydroxide, aluminum phosphate, aluminum potassium sulfate, AS04, MF59, AS01_{B}, CpG 1018, and IVAX-1. In an alternate embodiment, the vaccine preparation does not comprise an adjuvant. In another embodiment, the extracellular blebs comprise one or more of the following surface and maturation markers CD11c, MHC I, CD40, CD80, and/or CD86.

The disclosure further provides a method of making the vaccine preparation of the disclosure, comprising: generating extracellular blebs from a hybridoma by contacting the hybridoma with the one or more sulfhydryl blocking agents for 30 min to 24 h; isolating the extracellular blebs. In a further embodiment, the one or more sulfhydryl blocking agents are selected from the group consisting of mercury chloride, p-chloromercuribenzene sulfonic acid, auric chloride, p-chloromercuribenzoate, chlormerodrin, meralluride sodium, iodoacetmide, paraformaldehyde, dithiothreitol, and N-ethylmaleimide. In yet a further embodiment, the one or more sulfhydryl blocking agents is N-ethylmaleimide or paraformaldehyde. In another embodiment, N-ethylmaleimide is used at a concentration of 0.2 mM to 30 mM, or paraformaldehyde is used at a concentration of 10 mM to 100 mM.

The disclosure also provides a method of immunizing a subject in need thereof, comprising administering a therapeutically effective amount of the vaccine preparation disclosed herein. In a further embodiment, the vaccine preparation is administered intramuscularly, subcutaneously, intradermally, or intratumorally. In a further embodiment, the subject has cancer, an autoimmune disease, a neurodegenerative disorder, or an infection by a pathogen.

### BRIEF DESCRIPTION OF THE DRAWINGS

FIG. 1 presents an overview illustration of preparing EBs derived from the hybridomas of a bone marrow-derived dendritic cells (BMDC) at a desired maturation status and a T lymphoma cell with or without a distinct antigen. Briefly, bone marrow isolated from C57BL/6 mice were differentiated to BMDCs and further matured by the incubation with LPS. Both immature and mature BMDCs (iDCs and mDCs, respectively) were then fused with E.G7-OVA-GFP (E7OG) or EL4 (E4) T cell lymphoma cells with or without a distinct antigen (i.e., OVA), respectively, via polyethylene glycol (PEG). The resulting hybridoma cells were sorted by flow cytometry and cloned in a 96 well plate to obtain a pure population. Finally, EBs from the hybridomas with varying DC maturation statuses with or without OVA antigen were prepared by chemically induced blebbing and a series of centrifugation.

FIG. 2 provides images of BMDC-EG7-Ova-GFP hybridoma cells grown in media and semi-solid medium. The resulting clones were analyzed by CD11c (dendritic cell marker) and GFP using flow cytometry.

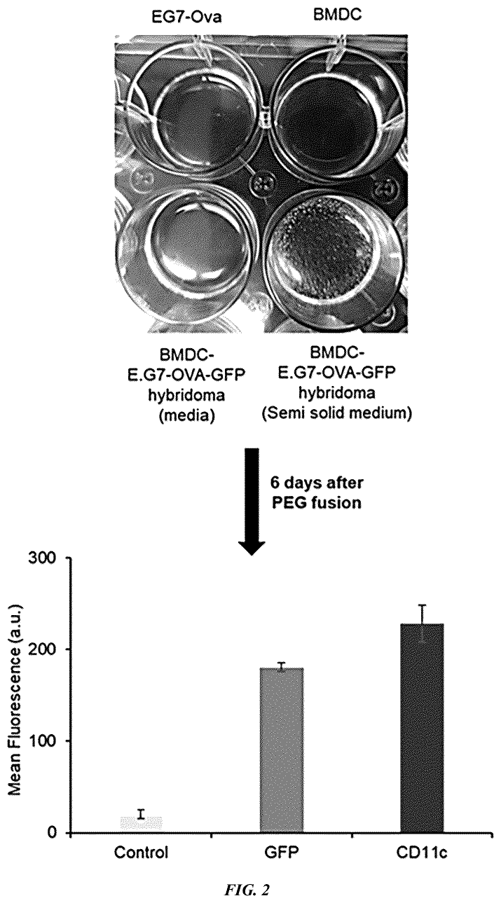

FIG. 3A-B provides for the preparation of pure DC/tumor hybridomas and their stability in presenting surface molecules over passages. (A) The PEG-mediated fusion resulted in ˜40% mDC/E7OG hybridoma cells which were further sorted and cloned to be a pure population, as quantified by flow cytometry. The resulting hybridomas were analyzed for their GFP expression using microscopy prior to sorting (left) and after sorting (right). (B) imDC/E7OG and mDC/E7OG were expanded up to 40 passages and their biological stability was analyzed by flow cytometry for CD11c, CD40, CD80, CD86, MHC I, MHC II, and MHC I/SIINFEKL. The hybridomas maintained their immunostimulatory statuses stemmed from the parent DCs regardless of passages.

FIG. 4 provides immature and mature hybridomas that were labeled with an anti-CD11c, CD40, CD80, CD86, MHC I, or MHC II antibodies and analyzed using flow cytometry.

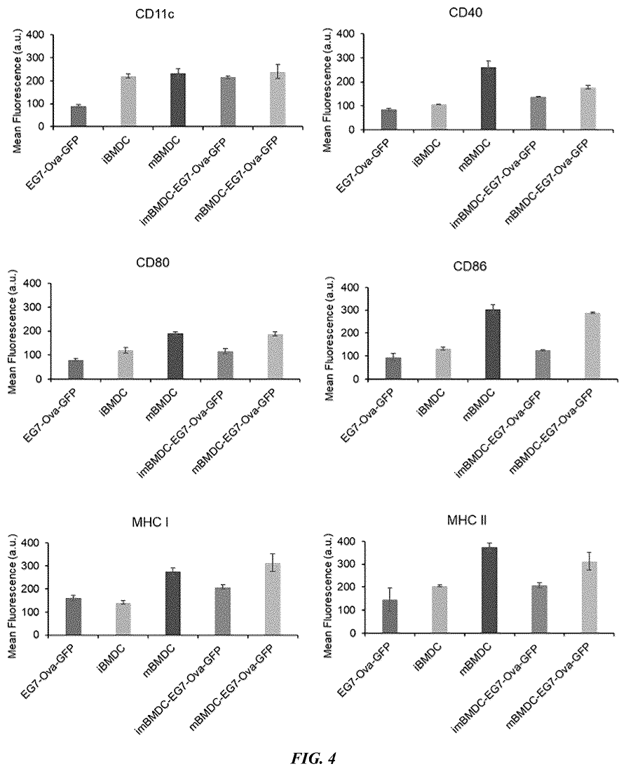

FIG. 5A-C presents an analysis of hybridomas of a bone marrow-derived dendritic cells (BMDC) at varying maturation statuses fused with EL4 T lymphoma cells. Briefly, bone marrow isolated from C57BL/6 mice were differentiated to BMDCs in the presence of GM-CSF and further matured by the incubation with LPS. (A) Both immature and mature BMDCs (iDCs and mDCs, respectively) fused with EL4 (E4) T lymphoma cells without a distinct antigen via polyethylene glycol (PEG)-mediated cell fusion. The resulting hybridomas retained the CD11c marker, a DC marker. (B) The DC/E4 hybridomas were sorted and cloned in a 96 well plate in order to obtain a relatively pure population that express both CD11c (a DC marker) and CD90.2 (a marker for EL4 cells). (C) Both imDC/E4 and mDC/E4 hybridomas exhibited the maturation characteristics of the parent DCs by having CD11c, CD40, CD80, CD86, MHC I, and MHC II.

FIG. 6 shows immature and mature hybridoma cell lines that were cryopreserved, thawed and grown in specialized hybrid media. The hybridomas were cultured for three days and analyzed for positive clones using CD11c and GFP.

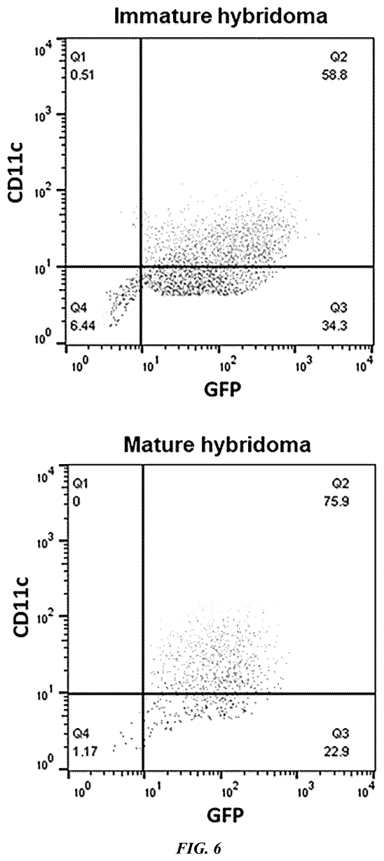

FIG. 7 shows immature hybridoma cells that were analyzed for CD11c, CD40, CD80, CD86, MHC I, MHC II, and MHC I-SIINFEKL after every 10 passages.

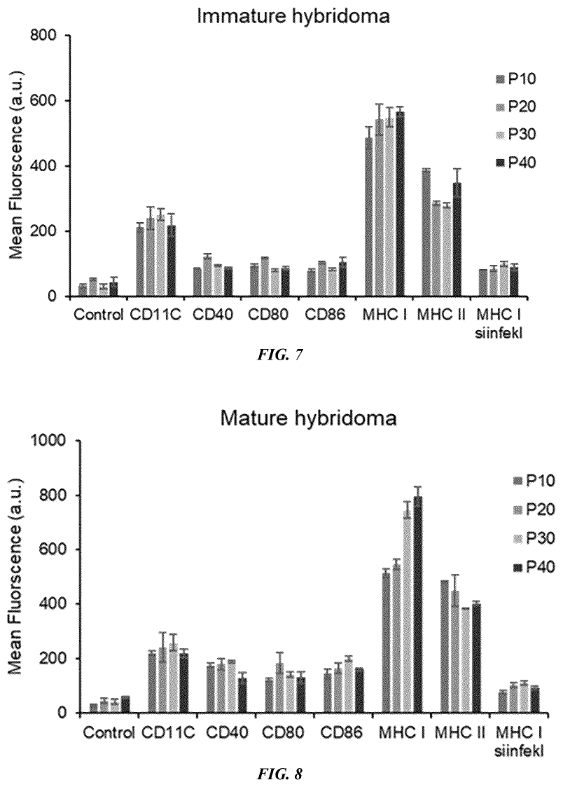

FIG. 8 shows mature hybridoma cells that were analyzed for CD11c, CD40, CD80, CD86, MHC I, MHC II, and MHC I-SIINFEKL after every 10 passages.

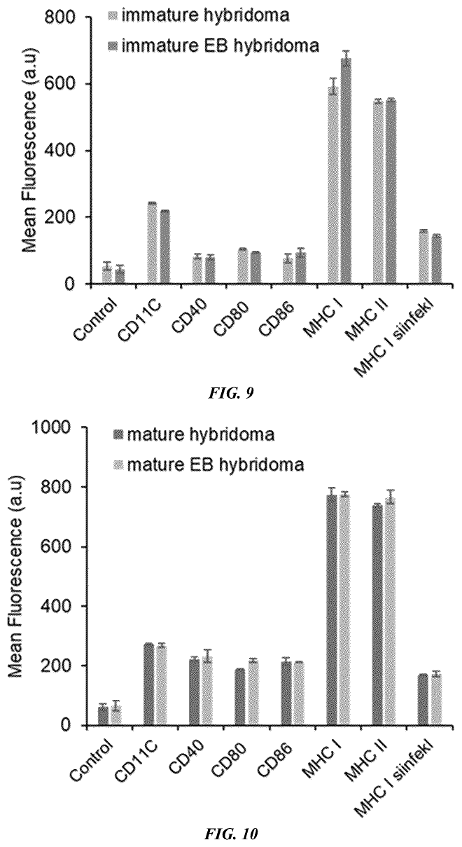

FIG. 9 presents immature hybridomas and blebs induced therefrom, which were analyzed for surface molecular presentation using flow cytometry.

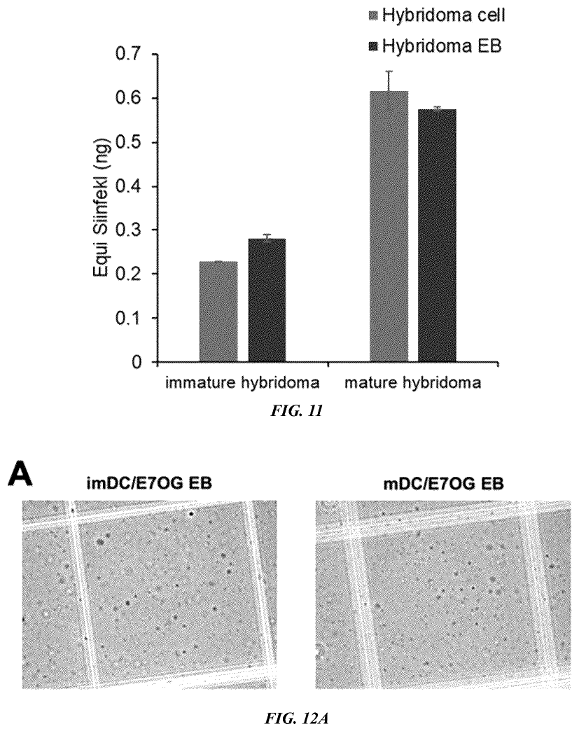

FIG. 10 presents immature hybridoma and blebs induced therefrom, which were analyzed for surface molecular presentation using flow cytometry

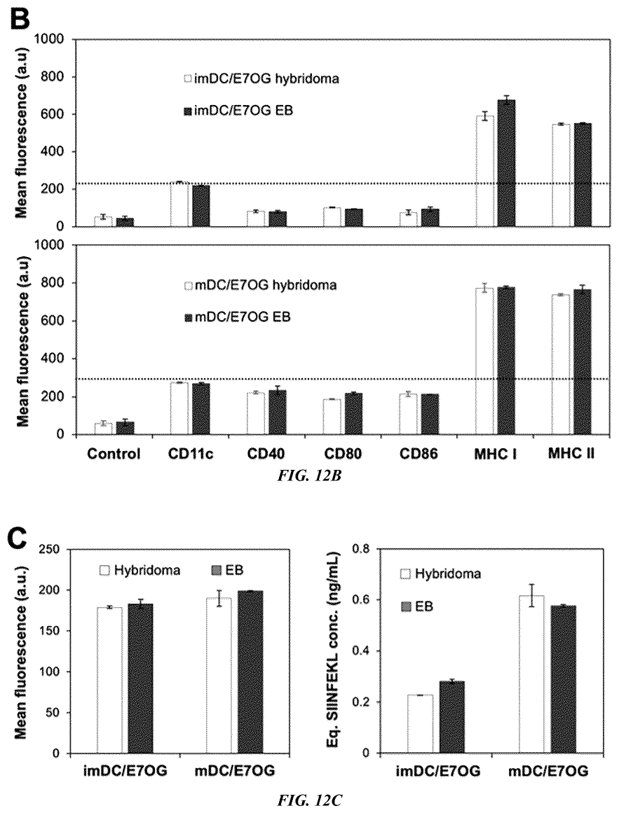

FIG. 11 demonstrates activation of CD8 T B3Z cells by immature and mature hybridomas and EBs induced therefrom, which were quantified by β-gal secretion.

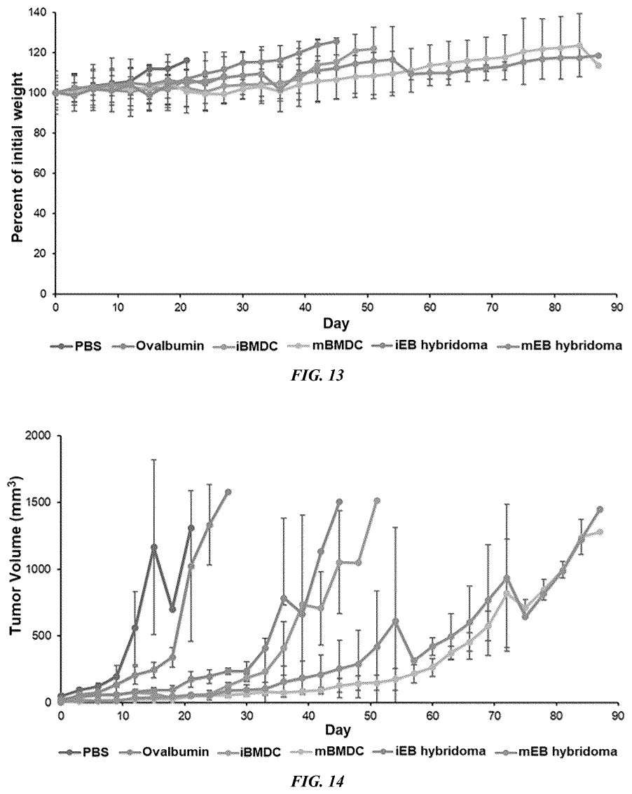

FIG. 12A-C demonstrates the retention of the surface molecular profile of dendritic cells and the resulting T cell activation by the corresponding EBs. (A) Morphology of EBs derived from imDC/E7OG and mDC/E7OG via incubation with NEM for 8 h, followed by a series of centrifugation to differentially remove cell debris and collect purified EBs. (B) EBs derived from imDC/E7OG and mDC/E7OG hybridomas were conformed to carry the same surface molecules of the corresponding parent hybridoma, as confirmed by flow cytometry for CD11c, CD40, CD80, CD86, MHC I, and MHC II. (C) The T cell stimulation capability of the parent DCs was closely translated to the corresponding EBs, as confirmed by flow cytometry for MHC I/SIINFEKL and CPRG assay of activated B3Z CD8 T hybridoma cells. The relative T cell activation was by compared by the SIINFEKL concentrations required for the same colorimetric signals generated by BMDCs incubated with SIILFEKL at varying concentrations.

FIG. 13 shows the body weight of mice vaccinated with PBS, Ova, iBMDC, mBMDC, or immature and mature EB hybridomas.

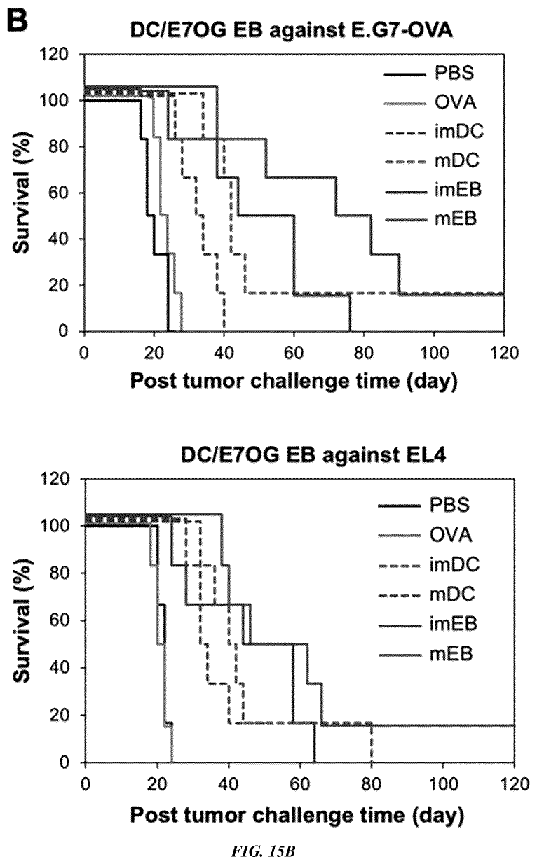

FIG. 14 presents the tumor volume of mice vaccinated with PBS, Ova, iBMDC, mBMDC, or immature and mature EB hybridomas.

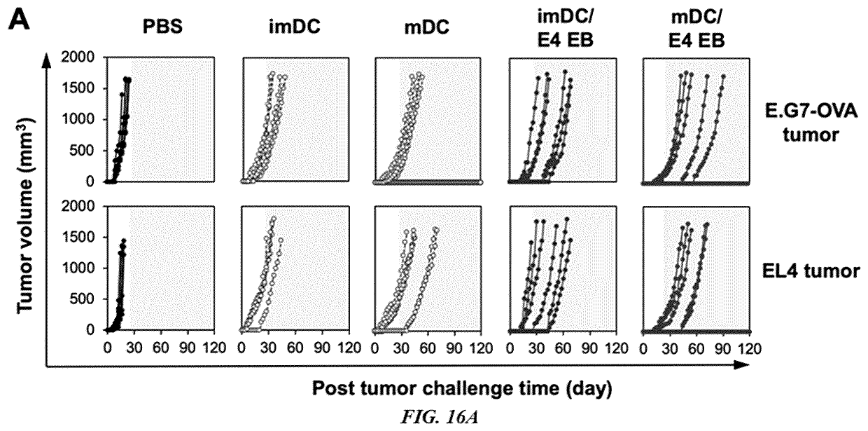

FIG. 15A-B shows the protection of mice from tumors when vaccinated with DC/E7OG EBs. (A) C57BL/6 mice were vaccinated with EBs or and DCs at varying immunostimulation with or without OVA, twice 14 days apart before the establishment of E7OG or E4 tumor 10 days after the second injection. The tumor volume was measured every 2 days and the mice carrying a tumor reaching 1.5 cm in diameter or larger were euthanized. (B) Survival plots of the vaccinated mice, as shown in A. Data are represented as mean±SD (n=6). Survival graph was analyzed by log-rank (Mantel Cox) test, **P<0.01, ***P<0.001 were considered significant. The survival statistics can be found in Tables 1 and 2.

FIG. 16A-B presents the protection of mice from tumors when vaccinated with DC/E4 EBs. (A) C57BL/6 mice were vaccinated with EBs or and DCs at varying immunostimulation with or without OVA, twice 14 days apart before the establishment of E7OG or E4 tumor 10 days after the second injection. The tumor volume was measured every 2 days and the mice carrying a tumor reaching 1.5 cm in diameter or larger were euthanized. (B) Survival plots of the vaccinated mice, as shown in A. Data are represented as mean±SD (n=6). Survival graph was analyzed by log-rank (Mantel Cox) test, **P<0.01, ***P<0.001 were considered significant. The survival statistics can be found in Tables 3 and 4.

FIG. 17A-B demonstrates the protection of mice from tumors when vaccinated with E.G7-OVA and EL4 tumor-derived EBs. (A) Tumor growth in C57BL/6 mice vaccinated with EBs derived from E.G7-OVA-GFP and EL4, twice 14 days apart before the establishment of the corresponding tumor 10 days after the second injection. (B) Survival plots of the mice in A. Data are represented as mean±SD (n=6).

FIG. 18A-B provides representative CTL activation plots in mice vaccinated with DC/E7OG EBs against E7OG or E4 tumor. (A) Specific lysis of E7OG tumor by the splenocytes harvested from mice vaccinated DC/E7OG EBs or DCs at varying maturation statuses. (B) Specific lysis of E4 tumor by the splenocytes harvested from mice vaccinated DC/E7OG EBs or DCs at varying maturation statuses. The splenocytes were harvested from the mice (n=5) 10 days after the second injection and incubated with the tumor cells for 4 h at an effector (splenocyte) to target (tumor) ratio of 25:1 and the specific lysis was determined by the percentage of YO-PRO-1-positive cells by flow cytometry. The bar graph on FIG. 21 shows the average percentage of specific lysis.

FIG. 19A-B provides representative CTL activation plots in mice vaccinated with DC/E4 EBs against E7OG or E4 tumor. (A) Specific lysis of E7OG tumor by the splenocytes harvested from mice vaccinated DC/E4 EBs or DCs at varying maturation statuses. (B) Specific lysis of E4 tumor by the splenocytes harvested from mice vaccinated DC/E4 EBs or DCs at varying maturation statuses. The splenocytes were harvested from the mice (n=5) 10 days after the second injection and incubated with the tumor cells for 4 h at an effector (solenocyte) to target (tumor) ratio of 25:1 and the specific lysis was determined by the percentage of YO-PRO-1-positive cells by flow cytometry. The bar graph of FIG. 21 shows the average percentage of specific lysis.

FIG. 20 shows specific lysis of EL4 and E.G7-OVA cancer cells at ratio's ranging from 100:1, 50:1, 25:1, 12.5:1, and 6.25:1.

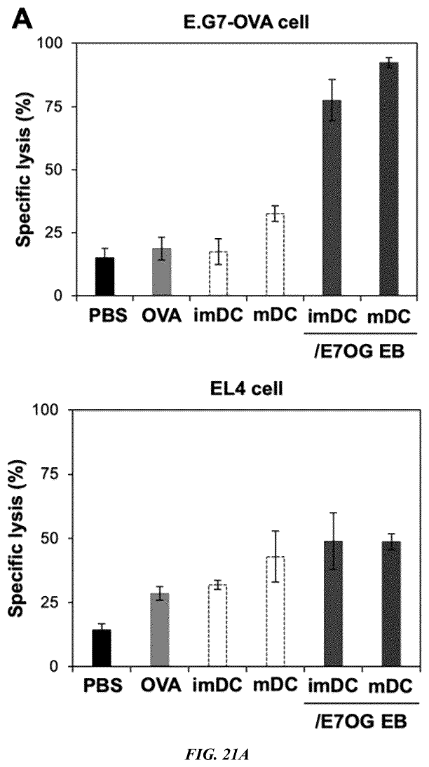

FIG. 21A-B shows the quantified activation of splenocytes harvested from the mice vaccinated with DCs and DC/tumor EBs against E.G7-OVA or EL4 tumor. (A) Specific lysis of E.G7-OVA and EL4 cells by the splenocytes harvested from the mice vaccinated with DCs and DC/E7OG EBs at varying maturation statuses. (B) Specific lysis of E.G7-OVA and EL4 cells by the splenocytes harvested from the mice vaccinated with DCs and DC/E4 EBs at varying maturation statuses. C57BL/6 mice were vaccinated twice with DCs and EBs 14 days apart and the splenocytes were harvested days after the second injection, prior to the incubation with the tumor cells for 4 h at an effector (splenocyte) to target (tumor cell) ratio of 25:1. The specific lysis of the tumor cells was determined by then ratio of YO-PRO-1-stained cells by flow cytometry and data are represented as mean±SD (n=6).

### DETAILED DESCRIPTION

As used herein and in the appended claims, the singular forms “a,” “an,” and “the” include plural referents unless the context clearly dictates otherwise. Thus, for example, reference to “an extracellular bleb” includes a plurality of such extracellular blebs and reference to “the vaccine” includes reference to one or more vaccines and equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of”

Unless defined otherwise, all technical and scientific terms used herein have the same meaning as commonly understood to one of ordinary skill in the art to which this disclosure belongs. Although many methods and reagents are similar or equivalent to those described herein, the exemplary methods and materials are disclosed herein.

All publications mentioned herein are incorporated herein by reference in full for the purpose of describing and disclosing the methodologies, which might be used in connection with the description herein. Moreover, with respect to any term that is presented in one or more publications that is similar to, or identical with, a term that has been expressly defined in this disclosure, the definition of the term as expressly provided in this disclosure will control in all respects.

It should be understood that this disclosure is not limited to the particular methodology, protocols, and reagents, etc., described herein and as such may vary. The terminology used herein is for the purpose of describing particular embodiments only and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used to described the present invention, in connection with percentages means±1%.

The terms “blebbing”, “plasma membrane blebbing” or “cell membrane blebbing” as used herein, all refer to methods disclosed herein that induce plasma membrane blebbing in cells resulting in the production of extracellular blebs (EBs). A bleb is an irregular bulge in the plasma membrane of a cell caused by localized decoupling of the cytoskeleton from the plasma membrane. The bulge eventually separates from the parent plasma membrane taking part of the cytoplasm with it to form a vesicle, i.e., an extracellular bleb. Blebbing is also involved in some normal cell processes, including cell locomotion and cell division. While typical blebbing of the plasma membrane is a morphological feature of cells undergoing late-stage apoptosis, chemically or physically induced blebbing is an active way to convert the plasma membranes at any cellular stages with preserved cellular and molecular profiles and consistently high yields. As such, cell blebbing can be manipulated by physical or chemical treatment. It can be induced following microtubule disassembly, by inhibition of actin polymerization, increasing membrane rigidity or inactivating myosin motors, and by modulating intracellular pressure. EBs can also be produced in response to various extracellular chemical stimuli, such as exposure to agents that bind up sulfhydryl groups (i.e., sulfhydryl blocking agents).

The term “blebbing agent”, as used herein refers to chemical agents, such as sulfhydryl blocking agents, that when administered to cells, induce the cells to undergo plasma membrane blebbing.

The term “extracellular bleb” or “EB” as used herein, is synonymous with an “induced cell-derived vesicle” or “ICV,” and refers to an extracellular vesicle that is formed as a direct result from contacting the cell with a blebbing agent. Accordingly, an EB is not synonymous with a naturally occurring extracellular vesicle, as the latter is formed without the presence of blebbing agent, while the former requires the use of a blebbing agent in order to be produced. The methods and compositions described herein can be applied to EBs of all sizes. In a particular embodiment, the method and compositions described herein comprise EBs that have an average diameter of 10 nm, 20 nm, 30 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, 950 nm, 1000 nm, 1100 nm, 1200 nm, 1300 nm, 1400 nm, 1500 nm, 1600 nm, 1700 nm, 1800 nm, 1900 nm, 2000 nm, 2500 nm, 3000 nm, 3500 nm, 4000 nm, 5000 nm, 10 μm, 15 μm, 20 μm, 30 μm, 40 μm, 50 μm, 60 μm, 70 μm, 80 μm, 90 μm, 100 μm, or any range that includes or is between any two of the foregoing values, including fractional increments thereof. Moreover, the EBs disclosed herein are produced from cells of interest that express an antigen that can modulate a subject's immune system. The EBs of the disclosure may further encapsulate biological molecules, such as nucleic acids, proteins, peptides, lipids, oligosaccharides, etc.; therapeutic agents, such as drug products like antivirals, antibiotics, and antifungals; prodrugs; gene silencing agents; chemotherapeutics; diagnostic agents; and components of a gene editing system, such as the CRISPR-Cas system, a CRISPRi system, or CRISPR-Cpf1 system, etc. In a particular embodiment, an EB disclosed herein is produced from a genetically engineered antigenic presenting cell or an infected cell that expresses foreign antigen(s) as disclosed herein, wherein the EB further comprises an encapsulated antiviral agent, antibiotic, and/or chemotherapeutic agent.

The term “nanometer sized extracellular bleb,” “nEB” or “nICV” as used herein, refer to EBs produced from cells using a blebbing agent as described herein having a dimeter in the nanometer size range. In a particular embodiment, the nEB has a diameter of 5 nm, 10 nm, 20 nm, 30 nm, 40 nm, 50 nm, 60 nm, 70 nm, 80 nm, 90 nm, 100 nm, 150 nm, 200 nm, 250 nm, 300 nm, 400 nm, 500 nm, 600 nm, 700 nm, 800 nm, 900 nm, up to 1000 nm, or is a range that includes or is between any two of the foregoing values, including fractional increments thereof.

The term “micrometer sized extracellular bleb”, or “mEB” or “mICV” as used herein, all refer to extracellular blebs produced from cells of interest using a blebbing agent as described herein having a dimeter in the micrometer size range. In a particular embodiment, the mEB has a diameter of 1 μm, 2 μm, 3 μm, 4 μm, 5 μm, 10 μm, 15 μm, 20 μm, 30 μm, 40 μm, 50 μm, 60 μm, 70 μm, 80 μm, 90 μm, 100 μm, or a range that includes or is between any two of the foregoing values, including fractional increments thereof.

The term “foreign antigen” as used herein, refers to an antigen that originates from outside the body. Examples of foreign antigens include parts of or substances produced by viruses or microorganisms (such as bacteria and protozoa), as well as substances in snake venom, certain proteins in foods, and components of serum and red blood cells from other individuals.

The term “endogenous antigen” refers to an antigen that originate from the subject's own cells. Examples of endogenous antigens include, but are not limited to, cancer or tumor antigens, and cellular antigens produced in result to an infection by a pathogen (e.g., bacteria, viruses, or fungi).

The term “sulfhydryl blocking agent” as used herein, refers to compound or reagent that interacts with cellular sulfhydryl groups so that the sulfhydryl group is blocked or bound up by the sulfhydryl blocking agent, typically via alkylation or disulfide exchange reactions. Chemical agents that can be used in the methods or compositions disclosed herein that block or bind up sulfhydryl groups include, but are not limited to, mercury chloride, p-chloromercuribenzene sulfonic acid, auric chloride, p-chloromercuribenzoate, chlormerodrin, meralluride sodium, iodoacetamide, paraformaldehyde, dithiothreitol and N-ethylmaleimide.

For purposes of the disclosure the term “cancer” will be used to encompass cell proliferative disorders, neoplasms, precancerous cell disorders and cancers, unless specifically delineated otherwise. Thus, a “cancer” refers to any cell that undergoes aberrant cell proliferation that can lead to metastasis or tumor growth. Exemplary cancers include but are not limited to, adrenocortical carcinoma, AIDS-related cancers, AIDS-related lymphoma, anal cancer, anorectal cancer, cancer of the anal canal, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, urinary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodermal tumors, visual pathway and hypothalamic glioma, breast cancer, including triple negative breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Seziary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, ocular cancer, islet cell tumors (endocrine pancreas), Kaposi Sarcoma, kidney cancer, renal cancer, laryngeal cancer, acute lymphoblastic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, AIDS-related lymphoma, non-Hodgkin lymphoma, primary central nervous system lymphoma, Waldenstram macroglobulinemia, medulloblastoma, melanoma, intraocular (eye) melanoma, merkel cell carcinoma, mesothelioma malignant, mesothelioma, metastatic squamous neck cancer, mouth cancer, cancer of the tongue, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, ewing family of sarcoma tumors, soft tissue sarcoma, uterine cancer, uterine sarcoma, skin cancer (non-melanoma), skin cancer (melanoma), papillomas, actinic keratosis and keratoacanthomas, merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and Wilm's Tumor.

The term “effective amount” as used herein, refers to an amount that is sufficient to produce at least a reproducibly detectable amount of the desired result or effect. An effective amount will vary with the specific conditions and circumstances. Such an amount can be determined by the skilled practitioner for a given situation.

The terms “patient”, “subject” and “individual” are used interchangeably herein, and refer to an animal, particularly a human, to whom treatment including prophylaxis treatment (e.g., vaccination) is provided. This includes human and non-human animals. The term “non-human animals” as used herein includes all vertebrates, e.g., mammals, such as non-human primates, (particularly higher primates), sheep, dog, rodent (e.g., mouse or rat), guinea pig, goat, pig, cat, rabbits, cows, and non-mammals such as chickens, amphibians, reptiles etc. In one embodiment, the subject is human. In another embodiment, the subject is an experimental animal or animal substitute as a disease model. “Mammal” refers to any animal classified as a mammal, including humans, non-human primates, domestic and farm animals, and zoo, sports, or pet animals, such as dogs, cats, cattle, horses, sheep, pigs, goats, rabbits, etc. Patient or subject includes any subset of the foregoing, e.g., all of the above, but excluding one or more groups or species such as humans, primates or rodents. A subject can be male or female. A subject can be a fully developed subject (e.g., an adult) or a subject undergoing the developmental process (e.g., a child, infant or fetus).

The term “isolated” when used in reference to an EB disclosed herein, refers to the fact that the EB is separated from most other cellular components from which it was generated or in which it is typically present in nature. The EBs disclosed herein are typically prepared to the state where they are substantially isolated to completely isolated from most other cellular components and cellular debris.

The term “therapeutically effective amount” as used herein, refers to an amount that is sufficient to affect a therapeutically significant reduction in one or more symptoms of the condition when administered to a typical subject who has the condition. A therapeutically significant reduction in a symptom is, e.g., about 10%, about 20%, about 30%, about 40%, about 50%, about 60%, about 70%, about 80%, about 90%, about 100%, or more as compared to a control or non-treated subject.

The term “treat” or “treatment” as used herein, refers to a therapeutic treatment wherein the object is to eliminate or lessen symptoms. Beneficial or desired clinical results include, but are not limited to, elimination of symptoms, alleviation of symptoms, diminishment of extent of condition, stabilized (i.e., not worsening) state of condition, delay or slowing of progression of the condition.

Immunotherapy has been of great interest as one of the most promising and diverse therapeutic strategies for a broad range of infectious diseases and cancers. Activation the immune system by delivering an immunologically active materials (e.g., antigens), commonly called vaccines, has made substantial contributions to improving human health and welfare. Successful vaccination is centrally played by antigen presenting cells (APCs) that present target antigenic peptides to T cells with immunostimulatory signals. Dendritic cells (DC) are professional APCs with exceptionally potent capability of activating T cells, making them a prime target for cell-based vaccination, especially against cancer. Autologous

Antigen presenting cells (APCs) or accessory cells are cells that display antigen bound by major histocompatibility complex (MHC) proteins on its surface; this process is known as antigen presentation. T cells may recognize these complexes using their T cell receptors (TCRs). APCs process antigens and present them to T-cells. The APC involved in activating T cells is usually a dendritic cell. T cells cannot recognize (and therefore cannot respond to) “free” or soluble antigens. They can only recognize and respond to antigen that has been processed and presented by cells via carrier molecules like MHC molecules. Helper T cells can recognize exogenous antigen presented on MHC class II; cytotoxic T cells can recognize endogenous antigen presented on MHC class I. Most cells in the body can present antigen to CD8+ cytotoxic T cells via MHC class I; however, the term “antigen-presenting cell” is often used specifically to describe professional APCs. Such cells express MHC class I and MHC class II molecules and can stimulate CD4+ helper T cells as well as cytotoxic T cells.

Professional APCs specialize in presenting antigens to T cells. They are very efficient at internalizing antigens, either by phagocytosis (e.g., macrophages), macropinocytosis, or by receptor-mediated endocytosis (B cells), processing the antigen into peptide fragments and then displaying those peptides (bound to a class I MHC molecule or to class II MHC molecule) on their membrane. The T cell recognizes and interacts with the antigen-class I MHC molecule complex or the antigen-class II MHC molecule complex on the membrane of the antigen-presenting cell. An additional co-stimulatory signal is then produced by the antigen-presenting cell, leading to activation of the T cell. The expression of co-stimulatory molecules and MHC class II are defining features of professional APCs. All professional APCs also express MHC class I molecules as well.

The main types of professional antigen-presenting cells are dendritic cells, macrophages and B cells.

Dendritic cells have the broadest range of antigen presentation and are necessary for activation of naïve T cells. DCs present antigen to both helper and cytotoxic T cells. They can also perform cross-presentation, a process by which they present exogenous antigen on MHC class I molecules to cytotoxic T cells. Cross-presentation allows for the activation of these T cells. Dendritic cells also play a role in peripheral tolerance, which contributes to prevention of auto-immune disease.

Prior to encountering foreign antigen, dendritic cells express very low levels of MHC class II and co-stimulatory molecules on their cell surface. These immature dendritic cells are ineffective at presenting antigen to T helper cells. Once a dendritic cell's pattern-recognition receptors recognize a pathogen-associated molecular pattern, antigen is phagocytosed and the dendritic cell becomes activated, upregulating the expression of MHC class II molecules. It also upregulates several co-stimulatory molecules required for T cell activation, including CD40 and B7. The latter can interact with CD28 on the surface of a CD4+ T cell. The dendritic cell is then a fully mature professional APC. It moves from the tissue to lymph nodes, where it encounters and activates T cells.

Dendritic cells (DC) can be found in practically all tissues, where they detect homeostatic imbalances and process antigens for presentation to T cells, establishing a link between innate and adaptive immune responses. Furthermore, DC can secrete cytokines and growth factors that modify ongoing immune responses, and are influenced by their interactions with other immune cells, like natural killer and innate lymphoid cells (ILCs).

Dendritic cells are found in two different functional states, “mature” and “immature”. These are distinguished by many features, but the ability to activate antigen-specific naïve T cells in secondary lymphoid organs is the hallmark of mature dendritic cells. Dendritic cell maturation is triggered by tissue homeostasis disturbances, detected by the recognition of pathogen-associated molecular patterns (PAMP) or damage-associated molecular patterns (DAMP). Maturation turns on metabolic, cellular, and gene transcription programs allowing dendritic cells to migrate from peripheral tissues to T-dependent areas in secondary lymphoid organs, where T lymphocyte-activating antigen presentation may occur.

During maturation, DC lose adhesive structures, reorganize the cytoskeleton and increase their motility. DC maturation also leads to a decrease in their endocytic activity but increased expression of MHC-II and co-stimulatory molecules. Mature DC express higher levels of the chemokine receptor, CCR7 and secrete cytokines, essential for T cell activation. Thus, the interaction between mature DC and antigen-specific T cells is the trigger of antigen-specific immune responses. When interacting with CD4+ T cells, DC may induce their differentiation into different T helper (Th) subsets such as Th1, Th2, Th17, or other CD4+ T cell subtypes. Immature DC are poor inducers of naïve lymphocyte effector responses, since they have low surface expression of co-stimulatory molecules, low expression of chemokine receptors, and do not release immunostimulatory cytokines. These “immature” cells, though, are very efficient in antigen capture due to their high endocytic capacity, via receptor-mediated endocytosis, including lectin-, Toll-like-, FC- and complement receptors and macropinocytosis. Thus, immature DC act, indeed, as sentinels against invading pathogens, but also as tissue scavengers, capturing apoptotic and necrotic cells.

Given their unique role in initiating primary immune responses, vaccine products prepared from patient-derived cultured DCs are under development for a broad range of vaccine applications, and are prepared from peripheral blood or from bone marrow. Such personalized DC based vaccines have shown to be safe and well-tolerated, and they induce antigen specific CD4+ and CD8+ T cell responses. However, efficacy of such vaccine products has been documented in only limited patient numbers and have not shown consistent clinical success. This is mainly attributable to the fact that the potency of patient-based products is impossible to standardize. It is possible to develop standard operating procedures for harvesting dendritic cell from blood or bone marrow and generating functional dendritic cells, but full reproducibility will always be hampered by patient-to-patient variability. Additionally, there are substantial hurdles regarding large scale implementation, time-consuming, and expensive. Furthermore, DCs often adapt themselves to the pathological microenvironments (e.g., becoming suppressed in tumor tissue).

One of the examples of highly demanded immunotherapies is cancer vaccines. Many strategies have been developed to load tumor antigens onto DCs for tumor vaccines. DCs pulsed with tumor-associated peptides or proteins result in the induction of anti-tumor immunity, introduction of tumor antigens into DCs through viral vectors encoding tumor-specific genes or through transfection with liposomal DNA or RNA. These strategies are effective to some degree but share certain drawbacks. For example, few tumor antigens have been identified and tumor cells may evade recognition through downregulation of a tumor antigen. As an alternative strategy, DCs fused to cancer cells through chemical, physical, or biological methods creates a heterokaryon that combines DC-derived co-stimulatory molecules, and the presence of specific tumor-derived antigens. DC-cancer fusion cells thus combine the essential elements for developing and presenting tumor antigens to immune cells and for inducing effective anti-tumor responses. However, the main limitation to the use of DC-cancer cell fusions is the availability of adequate amounts of autologous DC cells, limited availability of viable cancer samples, and low clinical responses. Although fusion technology is a versatile approach in the design of cancer vaccines and can be applicable to nearly all types of cancer cells, such versatility and wide applicability lead to variations that make it more difficult to standardize the vaccine. Most importantly, DC-cancer cell fusions have substantial safety concerns.

DCs loaded ex vivo with tumor-associated antigens (TAAs) have shown mostly prophylactic and some therapeutic efficacies in many pre-clinical studies as well as some early clinical trials. DCs are conventionally loaded with pre-determined TAAs via incubation tumor cell lysates, tumor-derived apoptotic bodies, purified TAA proteins, or TAA-derived peptides, or transduction with TAA-encoding cDNA or mRNA. However, this approach to employing a known antigen for cancer vaccination is challenged by lacking a distinct antigen in many forms of cancers, limited antigenic molecular profiles to be provided to the immune system, and time- and labor-intensive identification and preparation. Alternatively, hybridization of a DC and a tumor cell offers an advantage of ensured presentation of a full TAA repertoire, including unidentified antigens, to T naïve cells, as demonstrated in various animal models and cancer patients at an advanced stage. Fusing a DC with a tumor cell using a fusogenic chemical agent (e.g., polyethylene glycol [PEG]), electric field (electrofusion), or a virus inevitably produces unfused cells as well as fused cells of the same kinds, often requiring further purification for improved efficacy.

Despite the safety and confirmed immunogenicity, the outcomes of the clinical trials on cell-based vaccination against cancer have been largely disappointing. This is mainly attributable to the fact that the daunting potency standardization, reproducibility, and preparation scalability of patient-derived materials. In addition, adaption to the immune-suppressive tumor microenvironment and precarious storage are known to be a common challenge for cell-based cancer immunotherapy such as DC-based vaccines and CAR-T cell therapies. Extracellular vesicles (EVs) have been investigated as a cell-free alternative to cell-based immunotherapy, but EV-based therapeutics (e.g., DC-derived exosomes, dexosomes) have been slowly progressing to clinical translation due to high heterogeneity in both structure and function, low yield and purity, poor quality control, and limited scalability.

The disclosure provides compositions, preparations and methods to enhance the immunogenicity for cancer by fusion of an APC and a target cell to produce a stable hybridoma. Then optimally immune-activated hybridoma are converted to cell-free, cell-like vesicles via chemically and physically induced process which is rapid, efficient, and cost- and labor-friendly. The resulting extracellular blebs produced from the APC-target cell hybridoma can present and deliver antigenic proteins and present them in complex with MHC I and II molecules to activate the T cells with fully optimized molecular machineries in a desired mode of immune activation (or suppression).

The compositions, preparations and methods of the disclosure overcome major limitations related to the current vaccine formulation of proteins, nucleic acids, and dendritic cells, including molecularly tunable immune response, warranted immune response in a desired mode, comprehensive immune activation against a full library of antigens, the availability of adequate amounts of dendritic cells, quality control, safety, and efficacy. The fusion of DCs and target cells are more efficient and comprehensive in antigen presentation, creating immunogenic cells that induce potent immunity. In addition, engineering of DCs and target cells can be performed independently while each cell characteristic can persist for an extended period. The use of cell-free based APC-target cell vaccinations provide an excellent control mechanism for production of vaccine molecules that are safer and more efficacious than conventional vaccines including cell-based ones. Moreover, unlike cells, the cell-like, cell-free EBs are stable for storage, highly amendable to varying formulations, and capable of sustained T cell activation for an extended period time.

The key concept of the cancer immunotherapy is that the manipulation of the immune system can achieve cancer control and, ideally, cure. Many studies have shown clinical benefit when using general immune system activators, such as bacterial products and TLR agonists. The antitumor activity of these approaches, when it occurs, is attributed to the ability of these compounds to activate the immune system that, in turn, acquires the ability to kill tumor cells. Much of this effect was shown to be due to DC activation followed by the generation of T cell responses. Dendritic cells, as key activators of the adaptive immune response, would be expected to have a central role in inducing antitumor immune responses and the many functional deviations these cells show in cancer patients emphasize the relevant role they may, indeed, play in anti-tumor immune responses. In face of these data, it would be intuitive to exploit the immune activating potential of DC to induce antitumor responses in cancer patients. However, because of the difficulty of obtaining large numbers of these cells by non-invasive methods, therapeutic approaches using DC have been curtailed.

The disclosure provides compositions, preparations and methods that can obviate the DC production issues, by providing DC based hybridomas that can be grown up in large numbers, and further, can generate a near unlimited amount of EBs using the methods of the disclosure. In particular, the hybridomas of the disclosure have been generated from an antigen presenting cell and a target cell (e.g., cancer cell) in order to activate (e.g., cancer vaccine) or suppress (e.g., autoimmune disease) the immune system. The antigen presenting cells used for hybridoma formation can come from any source, ideally from humans; can include any subtype of antigen presenting cell (e.g., bone marrow-derived dendritic cells); and/or can include any maturation state of the antigen presenting cell (e.g., immature DCs or mature DCs). Additionally, the source of antigen presenting cells for the hybridoma can come from the subject to be treated, i.e., personalized treatment. In regards to the target cell, the target cell typically is a cancer cell. The type of cancer cell is not limiting, as most types of cancer cells can be fused with a dendritic cell using the methods described herein and those described in the art. Examples of cancer cells that can be used to be make a hybridoma of the disclosure include, but are not limited to, myelomas, lymphomas, carcinomas, sarcomas, leukemias, adenocarcinomas, thymomas, or other types of cancer cells.

The EB production technique disclosed herein presents a scalable option for producing cell-like, cell-free-based vaccines from hybridomas that have industrial and medicinal applicability. Moreover, by using the blebbing agents described herein, hybridomas can be induced to produce nano- and micro-scale EBs that can be used as vaccines. By maintaining the bioactive properties of the hybridomas, EBs can elicit an immune response when administered. In the studies presented herein, the EBs can elicit a strong immune response, even in the absence of adjuvants. Furthermore, the EBs disclosed herein can be loaded with other therapeutic agents (e.g., chemotherapeutics) or adjuvants, if so desired.

As shown in the studies presented herein, EBs that were produced from hybridomas had improved anticancer effects in vivo, compared to the hybridomas themselves. Based upon these results, the compositions, methods, and kits of the disclosure find use as presenting and delivering antigenic proteins and presenting them in complex with MHC class I and class II molecules to activate T Cells. The potential advantages of the compositions, methods and kits of the disclosure include, but are not limited to, enabling modulation of the immune responses to produce more effective type of immunity for cancer and foreign antigens, stable for storage, highly amendable to varying formulations, and capable of sustained T cell activation for an extended period time in the body.

In particular, the disclosure provides for techniques and methods that provide for high yields of EBs from hybridomas in as little as a few hours, producing both micro and nano-scale sized EBs. For example, use of the blebbing agents described herein can induce the production of EBs in as little as 30 min, optimized to 8 h in the studies presented herein.

In a further embodiment, the chemical agent that induces blebbing is a sulfhydryl blocking agent. Examples of sulfhydryl blocking agents include, but are not limited to, mercury chloride, p-chloromercuribenzene sulfonic acid, auric chloride, p-chloromercuribenzoate, chlormerodrin, meralluride sodium, iodoacetmide, paraformaldehyde, dithiothreitol, and N-ethylmaleimide. In a particular embodiment, EBs are produced from blebbing induced in hybridomas with (1) paraformaldehyde, (2) paraformaldehyde and dithiothreitol, or (3) N-ethylmaleimide. In a further embodiment, EBs are produced from blebbing induced in hybridomas by contacting the hybridomas with a solution comprising paraformaldehyde at of 5 mM, 10 mM, 15 mM, 20 mM, 25 mM, 30 mM, 35 mM, 40 mM, 50 mM, 55 mM, 60 mM, 65 mM, 70 mM, 75 mM, 80 mM, 85 mM, 90 mM, 95 mM, 100 mM, 110 mM, 120 mM, 130 mM, 140 mM, 150 mM, 160 mM, 170 mM, 180 mM, 190 mM, 200 mM, 210 mM, 220 mM, 230 mM, 240 mM, 250 mM, or a range that includes any two of the foregoing concentrations, including from 10 mM and 100 mM, and from 20 mM to 50 mM.

In a yet a further embodiment, the solution comprising paraformaldehyde (PFA) further comprises dithiothreitol (DTT) at concentration of 0.2 mM, 0.4 mM, 0.5 mM, 0.6 mM, 0.8 mM, 1 mM, 1.1 mM, 1.2 mM, 1.3 mM, 1.4 mM, 1.45 mM, 1.5 mM, 1.55 mM, 1.6 mM, 1.65 mM, 1.7 mM, 1.75 mM, 1.8 mM, 1.85 mM, 1.9 mM, 1.95 mM, 2.0 mM, 2.1 mM, 2.2 mM, 2.3 mM, 2.4 mM, 2.45 mM, 2.5 mM, 2.55 mM, 2.6 mM, 2.65 mM, 2.7 mM, 2.75 mM, 2.8 mM, 2.85 mM, 2.9 mM, 2.95 mM, 3.0 mM, 3.1 mM, 3.2 mM, 3.3 mM, 3.4 mM, 3.45 mM, 3.5 mM, 3.55 mM, 3.6 mM, 3.65 mM, 3.7 mM, 3.75 mM, 3.8 mM, 3.85 mM, 3.9 mM, 3.95 mM, 4.0 mM, 4.5 mM, 5.0 mM, 5.5 mM, 6.0 mM, 6.5 mM, 7.0 mM, 7.5 mM, 8.0 mM, 8.5 mM, 9.0 mM, 9.5 mM, 10 mM, or any range that includes or is between any two of the foregoing concentrations, including from 1.0 mM to 3 mM, and 1.5 mM to 2.5 mM. In an alternate embodiment, ICVs are produced from blebbing induced in hybridomas by contacting the hybridomas with a solution comprising N-ethylmaleimide (NEM) at concentration of 0.2 mM, 0.4 mM, 0.5 mM, 0.6 mM, 0.8 mM, 1.0 mM, 1.5 mM, 2.0 mM, 2.5 mM, 3.0 mM, 3.5 mM, 4.0 mM, 4.5 mM, 5.0 mM, 5.5 mM, 6.0 mM, 6.5 mM, 7.0 mM, 7.5 mM, 8.0 mM, 8.5 mM, 9.0 mM, 9.5 mM, 10.0 mM, 10.5 mM, 11.0 mM, 11.5 mM, 12 mM, 12.5 mM, 13.0 mM, 13.5 mM, 14.0 mM, 14.5 mM, 15.0 mM, 15.5 mM, 16.0 mM, 16.5 mM, 17.0 mM, 17.5 mM, 18.0 mM, 18.5 mM, 19.0 mM, 19.5 mM, 20.0 mM, or any range that includes or is between any two of the foregoing concentrations, including from 2.0 mM to 20.0 mM, and 2.0 mM to 5.0 mM. In a further embodiment, the solution comprising PFA; PFA and DTT; or NEM, comprises a buffered balanced salt solution. Examples of buffered saline solutions include but are not limited to, phosphate-buffered saline (PBS), Dulbecco's Phosphate-buffered saline (DPBS), Earles's Balanced Salt solution (ICVSS), Hank's Balanced Salt Solution (HBSS), TRIS-buffered saline (TBS), and Ringer's balanced salt solution (RBSS). In a further embodiment, the solution comprising PFA; PFA and DTT; or NEM comprises a buffered balanced salt solution at a concentration/dilution of 0.5×, 0.6×, 0.7×, 0.8×, 0.9×, 1×, 2×, 3×, 4×, 5×, 6×, 7×, 8×, 9×, and 10×, or any range that includes or is between any two of the foregoing concentrations/dilutions, including fractional values thereof.

In a certain embodiment, the disclosure also provides that the EBs may be collected by any suitable means to separate EBs from hybridomas or cell debris of hybridomas. In some embodiments, to isolate EBs, cells and cell debris can be removed by centrifugation at 1000 to 1500 rpm for 1, 1.5, 2, 2.5, 3, 3.5, 4, 4.5, 5, 5.5, 6, 6.5, 7, 7.5, 8, 8.5, 9, 9.5, or 10 minutes followed by removal of hybridomas and cell debris of hybridomas. mEBs and nEBs can then be recovered by centrifugation at 10,000×g to 18,000×g for 5, 6, 7, 8, 9, 10, 11, 12, 13, 14, or 15 minutes. EBs may be further concentrated using concentrators. The size of the EBs disclosed herein could be controlled by using the isolation methods presented herein.

The disclosure further provides that the EBs disclosed herein may be used (1) in combination with other agents or molecules, and/or (2) loaded with other agents or molecules, such as biological molecules, therapeutic agents, prodrugs, adjuvants, diagnostic agents, and/or components of gene editing systems. In a particular embodiment, the ICVs are used in combination with or loaded with a cargo comprising one or more chemotherapeutics.

EBs produced in accordance with embodiments of the disclosure may also be loaded with the cargo via direct membrane penetration, chemical labeling and conjugation, electrostatic coating, adsorption, absorption, electroporation, or any combination thereof. Further, EBs produced in accordance with certain embodiments of the disclosure may undergo multiple loading steps, such that some cargo may be introduced into the hybridomas prior to blebbing, while additional cargo may be loaded during or after blebbing. Additionally, EBs may be loaded with the cargo during blebbing, and further loaded with another cargo after blebbing. In a further embodiment, the EBs may be loaded with a cargo as defined above by incubating hybridomas or EBs with a cargo having the concentration of 25 μg/mL, 50 μg/mL, 100 μg/mL, 200 μg/mL, 300 μg/mL, 400 μg/mL, 500 μg/mL, 600 μg/mL, 700 μg/mL, 800 μg/mL, 900 μg/ml, 1 ng/mL, 10 ng/mL, 100 ng/mL, 1 μg/mL, 10 μg/mL or any range that includes or is between any two of the foregoing concentrations. Additionally, the incubation may occur for 1 hour, 2 hours, 3 hours, 4 hours, 5 hours, 6 hours, 12 hours, 24 hours, 48 hours, or any range that includes or is between any two of the foregoing time points. Alternatively, the loading conditions may occur at a ratio of ICVs to a compound of 1:20 to 20:1, 1:15 to 15:1, 12:1 to 1:12, 11:1 to 1:11, 10:1 to 1:10, 9:1 to 1:9, 8:1 to 1:8, 7:1 to 1:7, 6:1 to 1:6, 5:1 to 1:5, 4:1 to 1:4, 3:1 to 1:3, 2:1 to 1:2, 1.5:1 to 1:1.5, or 1:1. Additionally, the polydispersity of cargo-loaded EBs may have a similar polydispersity index (PDI) as unloaded ICVs. As such, cargo-loaded EBs may have a PDI of about 0.1, 0.2, 0.3, 0.4, 0.5, 0.6, 0.7, 0.8, 0.9, 1.0, or any range that includes or is between any two of the foregoing values.

The disclosure further provides for pharmaceutical compositions and formulations comprising EBs described herein for specified modes of administration. In one embodiment, a pharmaceutical composition comprises EBs and a pharmaceutically acceptable carrier. The term “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the composition and is compatible with administration to a subject, for example a human. Such compositions can be specifically formulated for administration via one or more of a number of routes, such as the routes of administration described herein. Supplementary active ingredients also can be incorporated into the compositions.

The disclosure further provides for the use of a pharmaceutical composition comprising EBs of the disclosure as cell-free vaccines for cancer, infectious diseases, and autoimmune disease. In a further embodiment, the disclosure also provides methods for immunizing a subject, comprising: administering an amount of EBs of the disclosure to elicit an immune response in the subject. Suitable methods of administering an EB preparation described herein to a patient include by any route of in vivo administration that is suitable for delivering EBs to a patient. The preferred routes of administration will be apparent to those of skill in the art, depending on the EB's preparation's type of vaccine used, and the target cell population. Preferred methods of in vivo administration include, but are not limited to, intravenous administration, intertumoral administration, intraperitoneal administration, intramuscular administration, intracoronary administration, intraarterial administration (e.g., into a carotid artery), subcutaneous administration, transdermal delivery, intratracheal administration, subcutaneous administration, intraarticular administration, intraventricular administration, inhalation (e.g., aerosol), intracerebral, nasal, oral, pulmonary administration, impregnation of a catheter, and direct injection into a tissue. In a particular embodiment, a preferred route of administration is by intramuscular injection of the EBs. In a further embodiment, a preferred route of administration is by intertumoral injection of the EBs. In yet a further embodiment, a preferred route of administration is by subcutaneous injection of the EBs.

Intravenous, intraperitoneal, subcutaneous, intradermal and intramuscular administrations can be performed using methods standard in the art. Aerosol (inhalation) delivery can also be performed using methods standard in the art (see, for example, Stribling et al., Proc. Nat. Acad. Sci. USA 189: 11277-11281, 1992, which is incorporated herein by reference in its entirety). Oral delivery can be performed by complexing an EB preparation of the present invention to a carrier capable of withstanding degradation by digestive enzymes in the gut of an animal. Examples of such carriers include plastic capsules or tablets, such as those known in the art.

The appropriate dosage and treatment regimen for the methods of vaccination described herein will vary with respect to the EBs being delivered, and the specific condition of the subject. In one embodiment, the administration is over a period of time until the desired effect is achieved (e.g., strong immune response to an infectious agent).

For use in the therapeutic applications described herein, kits and articles of manufacture are also described herein. Such kits can comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers can be formed from a variety of materials such as glass or plastic.

For example, the container(s) can comprise one or more EBs described herein, optionally in a composition or in combination with another agent as disclosed herein. The container(s) optionally have a sterile access port (for example the container can be an intravenous solution bag or a vial having a stopper pierceable by a hypodermic injection needle). Such kits optionally comprise a compound disclosed herein with an identifying description or label or instructions relating to its use in the methods described herein.

A kit will typically comprise one or more additional containers, each with one or more of various materials (such as reagents, optionally in concentrated form, and/or devices) desirable from a commercial and user standpoint for use of a compound described herein. Non-limiting examples of such materials include, but are not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific application. The label can also indicate directions for use of the contents, such as in the methods described herein.

The disclosure further provides that the methods, preparations and compositions described herein can be further defined by the following aspects (aspects 1 to 28):

1. A vaccine preparation comprising extracellular blebs from a hybridoma of an antigen presenting cell and a target cell, wherein the hybridoma expresses an antigen that can modulate a subject's immune system, wherein the extracellular blebs are produced from the hybridoma by treating the hybridoma with a blebbing agent, and wherein the antigen is displayed on the surface of the extracellular blebs, preferably wherein the antigen activates or the subject's immune system to the antigen, or alternatively, wherein the antigen deactivates the subject's immune system to the antigen, preferably wherein the vaccine preparation further comprises a pharmaceutically acceptable carrier, excipient, and/or diluent, preferably wherein the extracellular blebs are isolated extracellular blebs, and preferably wherein the extracellular blebs are from 1 μm to 500 μm in size.

2. The vaccine preparation of aspect 1, wherein the antigen presenting cell is selected from a macrophage, a B cell and a dendritic cell, preferably a dendritic cell.

3. The vaccine preparation of aspect 2, wherein the dendritic cell is a mature dendritic cell.

4. The vaccine preparation of aspect 2, wherein the dendritic cell is an immature dendritic cell.

5. The vaccine preparation of any one of aspects 2 to 4, wherein the dendritic cell is a bone marrow derived dendritic cell, a monocyte-derived dendritic cell, or a peripheral blood mononuclear cell-derived dendritic cell.

6. The vaccine preparation of any one of aspects 2 to 5, wherein the dendritic cell is a mammalian dendritic cell, preferably a human dendritic cell.

7. The vaccine preparation of any one of the proceeding aspects, wherein the target cell is selected from a cancer cell, an abnormal or diseased cell, a cell engineered to display an antigen, or a cell that is infected with an infectious agent, preferably wherein the target cell is a mammalian cell, more preferably wherein the target cell is a human cell.

8. The vaccine preparation of any one of the proceeding aspects, wherein the target cell is a cancer cell selected from a myeloma, a lymphoma, a carcinoma, a sarcoma, a leukemia, an adenocarcinoma, a thymoma, or a neoplastic cell from a malignant tumor.

9. The vaccine preparation of any one of the proceeding aspects, wherein the target cell is a cell that is infected by a virus, a fungus, or a bacterium.

10. The vaccine preparation of aspect 9, wherein the virus is selected from Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Coronavirus, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, Epstein-Barr virus, European bat lyssavirusalitis, GB virus C/Hepatitis G virus Pegivirus, Hantan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus, Human herpesvirus, Human immunodeficiency virus, Human papillomavirus, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Isfahan virus, JC polyomavirus, Japanese encephalitis virus, Junin arenavirus, KI Polyomavirus, Kunjin virus, Lagos bat virus, Lake Victoria Marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, Semliki forest virus, Seoul virus, Simian foamy virus, Simian virus, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus O, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, and Zika virus.

11. The vaccine preparation of aspect 9, wherein the fungus is selected from Absidia corymbifera, Absidia ramose, Achorion gallinae, Actinomadura spp., Ajellomyces dermatididis, Aleurisma brasiliensis, Allersheria boydii, Arthroderma spp., Aspergillus flavus, Aspergillus fumigatu, Basidiobolus spp., Blastomyces spp., Cadophora spp., Candida albicans, Cercospora apii, Chrysosporium spp., Cladosporium spp., Cladothrix asteroids, Coccidioides immitis, Cryptococcus albidus, Cryptococcus gattii, Cryptococcus laurentii, Cryptococcus neoformans, Cunninghamella elegans, Dematium wernecke, Discomyces israelii, Emmonsia spp., Emmonsiella capsulate, Endomyces geotrichum, Entomophthora coronate, Epidermophyton floccosum, Filobasidiella neoformans, Fonsecaea spp., Geotrichum candidum, Glenospora khartoumensis, Gymnoascus gypseus, Haplosporangium parvum, Histoplasma, Histoplasma capsulatum, Hormiscium dermatididis, Hormodendrum spp., Keratinomyces spp., Langeronia soudanense, Leptosphaeria senegalensis, Lichtheimia corymbifera, Lobmyces loboi., Loboa loboi, Lobomycosis, Madurella spp., Malassezia furfur, Micrococcus pelletieri, Microsporum spp., Monilia spp., Mucor spp., Mycobacterium tuberculosis, Nannizzia spp., Neotestudina rosatii, Nocardia spp., Oidium albicans, Oospora lactis, Paracoccidioides brasiliensis, Petriellidium boydii, Phialophora spp., Piedraia hortae, Pityrosporum furfur, Pneumocystis jirovecii (or Pneumocystis carinii), Pullularia gougerotii, Pyrenochaeta romeroi, Rhinosporidium seeberi, Sabouraudites (Microsporum), Sartorya fumigate, Sepedonium, Sporotrichum spp., Stachybotrys, Stachybotrys chartarum, Streptomyce spp., Tinea spp., Torula spp., Trichophyton spp., Trichosporon spp., and Zopfia rosatii.

12. The vaccine preparation of aspect 9, wherein the bacterium is selected from Actinomyces israelii, Bacillus anthracis, Bacillus cereus, Bartonella henselae, Bartonella quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Francisella tularensis, Haemophilus influenzae, Helicobacter pylori, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumoniae, Neisseria gonorrhoeae, Neisseria meningitidis, Pseudomonas aeruginosa, Rickettsia rickettsia, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Ureaplasma urealyticum, Vibrio cholerae, Yersinia pestis, Yersinia enterocolitica, and Yersinia pseudotuberculosis.

13. The vaccine preparation of any one of the preceding aspects, wherein the antigen presenting cell and/or target cell is from a subject that is to be treated with the vaccine preparation.

14. The vaccine preparation of any one of the preceding aspects, wherein the antigen comprises a tumor-specific antigen, or a tumor-associated antigen.

15. The vaccine preparation of aspect 14, wherein the tumor-specific antigen, or the tumor-associated antigen is selected from alphafetoprotein, carcinoembryonic antigen, CA-125, MUC-1, epithelial tumor antigen, tyrosinase, melanoma-associated antigen, k-ras, abnormal products of p53, alpha-actinin-4, ARTC1, B-RAF, BCR-ABL fusion protein, beta-catenin, CASP-5, CASP-8, CDc27, CDK12, CDK4, CDKN2A, CLPP, COA-1, COA-2, CSNK1A1, dek-can fusion protein, EFTUD2, Elongation factor 2, ETV6-AML1 fusion protein, FLT3-ITD, FN1, FNDC3B, GAS7, GPNMB, HAUS3, HLA-A11, HLA-A2, hsp70-2, LDLR-fucosyltransferaseAS fusion protein, MART2, MATN, MET, MUM-1, MUM-2, MUM-3, Myosin class I, N-ras, neo-PAP, NFYC, OGT, OS-9, p53, pml-RARalpha fusion protein, PPP1R3B, PRDX5, PTPRK, RBAF600, SIRT2, SNRPD1, SYT-SSX1 or -SSX2 fusion protein, TGF-betaRII, TP53, Triosephosphate isomerase, BAGE-1, CT37/FMR1NB, Cyclin-A1, D393-CD20n, GAGE-1,2,8, GAGE-3,4,5,6,7, GnTV, HERV-E, HERV-K-MEL, KK-LC-1, KM-H4N-1, LAGE-1, LRPAP1, LY6K, MAGE-A1, MAGE-A10, MAGE-A12m, MAGE-A2, MAGE-A3, MAGE-A4, MAGE-C1, MAGE-C2, mucin, NA88-A, NY-ESO-1/LAGE-2, SAGE, Sp17, SSX-2, SSX-4, TAG-1, TAG-2, TRAG-3, TRP2-INT2, XAGE-1b/GAGED2a, CEA, gp100/Pmel17, mammaglobin-A, Melan-A/MART-1, NY-BR-1, OA1, PAP, PSA, RAB38/NY-MEL-1, TRP-1/gp75, TRP-2, BCLX, BING-4, CALCA, CD274, CD45, CPSF, cyclin D1, DKK1, ENAH, epCAM, EphA3, EZH2, FGF5, glypican-3, HEPACAM, Hepsin, HER-2/neu, HLA-G, HSPH1, IGF2B3, IMP-3, MUC1, Meloe, Midkine, WTi, VEGF, TPBG, telomerase, STEAPI, STEAPI, RAGE-1, PSMA, PRAME, RGS5, RhoC, RNF43, RU2AS, SOX10, and survivin.

16. The vaccine preparation of any one of the proceeding aspects, wherein the antigen is a foreign antigen or a self-antigen.

17. The vaccine preparation of aspect 16, wherein the foreign antigen is from a bacterium, fungus or virus.

18. The vaccine preparation of aspect 16, wherein the self-antigen is selected from any biomolecule or a portion thereof that is found in the body of a subject, preferably where the self-antigen comprises a protein, a peptide or a portion thereof found in the body of a subject, preferably wherein the subject is a human subject.

19. The vaccine preparation of any one of the proceeding aspects, wherein the vaccine preparation further comprises an adjuvant.

20. The vaccine preparation of any one of the proceeding aspects, wherein the vaccine preparation does not comprise an adjuvant.

21. The vaccine preparation of any one of the proceeding aspects, wherein the extracellular blebs comprise one or more of the following surface and maturation markers CDI Ic, MHC I, CD40, CD80, and/or CD86, preferably wherein the extracellular blebs comprise CDI Ic, MHC I, CD40, CD80, and CD86 surface and maturation markers.

22. A method of making the vaccine preparation of any one of the proceeding aspects, comprising:
generating extracellular blebs from a hybridoma by contacting the hybridoma with the one or more sulfhydryl blocking agents for 30 min to 24 h;
isolating the extracellular blebs.

23. The method of aspect 22, wherein the one or more sulfhydryl blocking agents are selected from the group consisting of mercury chloride, p-chloromercuribenzene sulfonic acid, auric chloride, p-chloromercuribenzoate, chlormerodrin, meralluride sodium, iodoacetmide, paraformaldehyde, dithiothreitol, and N-ethylmaleimide.

24. The method of aspect 23, wherein the one or more sulfhydryl blocking agents is N-ethylmaleimide or paraformaldehyde.

25. The method of aspect 24, wherein N-ethylmaleimide is used at a concentration of 0.2 mM to 30 mM, or wherein paraformaldehyde is used at a concentration of 10 mM to 100 mM.

26. A method of immunizing a subject in need thereof, comprising administering a therapeutically effective amount of the vaccine preparation of any one of aspects 1 to 21 to the subject.

27. The method of aspect 26, wherein the vaccine preparation is administered intramuscularly, subcutaneously, intradermally, or intratumorally.

28. The method of aspect 26, wherein the subject has cancer, an autoimmune disease, a neurodegenerative disorder, or an infection by a pathogen.

The following examples are intended to illustrate but not limit the disclosure. While they are typical of those that might be used, other procedures known to those skilled in the art may alternatively be used.

### EXAMPLES

Cells and animals. EL4 murine T lymphoma cells were purchased from the ATCC (Manassas, VA) and cultured in Gibco™ DMEM supplemented with 10% (v/v) FBS, 2 mM L-glutamine, and 1% (w/v) penicillin-streptomycin (all from ThermoFisher Scientific, Waltham, MA). E.G7-OVA T lymphoma cells (ATCC), which were modified from EL4 cells to present ovalbumin-derived peptides, were cultured in RPMI hybrid media containing 10% FBS, 2 mM L-glutamine, 10 mM HEPES, 1 mM sodium pyruvate, 55 μM 2-mercaptoethanol, and 0.4 mg/mL geneticin, all from ThermoFisher. E.G7-OVA cells were further retrovirally transduced to express GFP as described by Shin et al. (Immune Network 16:134-139 (2016)), to make E.G7-OVA-GFP cells. B3Z CD8 T hybridoma cells that were engineered to produce β-galactosidase (LacZ) in response to SIINFEKL-loaded MHC I (H-2K^{b}) were cultured in the same media that was used to culture E.G7-OVA cells. Cells were passaged every 2 or 3 days and checked for Mycoplasma contamination using a Mycoplasma PCR Test Kit (ABM, Richmond, Canada). All cells were cultured at 37° C. with 5% CO₂ and 100% humidity. C57BL/6 mice (6-8-week-old, female, Charles River Laboratories, Wilmington, MA) were used in all animal studies, according to the animal use protocols (AUP-20-116) approved by IACUC of UC Irvine.

Bone marrow-derived dendritic cells (BMDCs). BMDCs were prepared as reported in Madaan et al. (Journal of Biological Methods 1(1):e1 (2014)) and Onai et al. (Methods Mol Biol 1423:53-9 (2016)). Briefly, the bone marrow isolated from euthanized mice was flushed out from the hind quarter femurs using a 27-gauge needle (ThermoFisher) filled with RPMI media. The bone marrow was then homogenized through a 40 μm sterile cell strainer (ThermoFisher), followed by the centrifugation of the collected cells at 300×g for 10 min at room temperature. After the supernatant was aspirated, the cell pellet was resuspended and incubated in 1 mL of 1×RBC lysis buffer (ThermoFisher) for 10 min at room temperature. After quenching the RBC lysis, 10 mL RPMI media was added to the mixture and centrifuged at 300×g for 10 min and the cell pellet was rinsed once by resuspending it in RPMI. After centrifugation, the cell pellet was resuspended in DC differentiation media consisting of RPMI media containing 10% FBS, 1% penicillin-streptomycin, and 20 ng/mL GM-CSF and the suspended cells harvested from a femur were plated in a 100 cm² cell culture dish (ThermoFisher). The media was supplemented with 10 mL of a freshly prepared media containing 20 ng/mL of GM-CSF after 2 days from the initial inoculation. In additional 3 days, the DC population was assessed by flow cytometry of the cells stained with anti-mouse CD11c antibody (Biolegend, San Diego, CA). After 5 days of differentiation to DCs in the presence of GM-CSF, loosely adherent cells were collected by gentle rinsing of the plates with a serological pipette and centrifugation at 300×g for 10 min. Collected DCs were incubated with RPMI media containing 10% FBS and 1% penicillin-streptomycin supplemented with 20 ng/mL lipopolysaccharide (LPS, Sigma Aldrich, St. Louis, MO) for 24 h in order to obtain mature DCs. Both immature and mature DCs were characterized for expression of surface molecules by flow cytometry.

Hybridomas of BMDC and T cell lymphoma cell. BMDCs were mixed with EL4 or E.G7-OVA-GFP cells at a ratio of 5:1, washed twice using serum free RPMI media at 300×g for 10 min and fused using a ClonalCell™ HY-Hybridoma kit (Stemcell technologies, Vancouver, Canada) following the manufacturer's procedures. After a 5-min incubation at room temperature, 10 mL of serum-free RPMI was slowly added to the fusion solution, and the cells were washed by centrifugation at 300×g for 10 min before incubating them in RPMI media containing GM-CSF at 20 ng/mL incubated at 37° C. for 6 days. The hybridoma of BMDC and T lymphoma cells were quantified by double-positive populations for CD11c (DC marker), GFP (E.G7-OVA-GFP cells), and CD90.2 (EL4 cells) by flow cytometry in 6 days and further sorted using a BD FACSAria™ Fusion cell sorter (BD Biosciences, Franklin Lakes, NJ). The cells were resuspended in PBS and analyzed for the cell surface molecules with dendritic cell characteristics. Briefly, 1×10⁶ cells were incubated with fluorescently labeled anti-mouse CD11c antibodies (Biolegend) for 20 min on ice and unbound antibodies were removed by centrifugation at 300×g for 10 min. The sorted hybridoma cells were further cloned by incubating 100 cells in a 96 well (ThermoFisher) (approximately a single cell per well) for 14 days. The clones with 90% or higher rate of simultaneous GFP expression or FITC-CD90.2 along with CD11c staining were collected and cultured in the hybrid culture media, as mentioned above. The hybridoma cells were passaged at a 1:20 ratio every 3 days and cryopreserved in 90% FBS and 10% DMSO for storage in liquid nitrogen. The stably maintained antigen presentation and maturation status of the hybridomas were confirmed by analyzing them by flow cytometry for CD11c, CD40, CD80, CD86, MHC I, and MHC II, and MHC I/SIINFEKL complexes after staining with the corresponding, fluorescently labeled antibodies (Biolegend), as described earlier, at every 10 cell passages until passage 40.

Polyethylene glycol (PEG) mediated fusion of bone marrow derived dendritic cells with EG7-Oova T cell lymphoma. BMDCs were obtained by incubating the bone marrow cells from C57BL/6 mice femurs, with 20 ng/mL GM-CSF for 5 days. The resulting BMDCs (˜80% CD11c+) were then incubated with or without 20 ng/mL LPS for 24 h, to prepare mBMDCs and immature DCs (iBMDCs), respectively. The BMDCs were fused with E.G7-OVA-GFP T lymphoma cells using polyethylene glycol (PEG) at a 5:1 ratio in serum free media. The fused cells were incubated for 15 minutes at 37° C. PEG was washed using serum free media at 300×g to 10 minutes for a total of two washes. The fused cells were placed in RPMI media containing 10% FBS, 1% pen-strep in presence of 20 ng/mL GM-CSF and cultured for 6 days prior to single cell sorting using CD11c (dendritic cell marker) and GFP by flow cytometry. The DC-EG7-ova-GFP hybridomas were maintained for growth in RPMI with 10% FBS, 1% pen-strep, 2 mM L-glutamine, 10 mM HEPES, 1.0 mM sodium pyruvate supplemented with 0.05 mM 2-mercaptoethanol, and 0.4 mg/mL G418. The resulting hybridoma showed a positive clone for CD11c, a representative DC marker and GFP as E.G7-OVA T lymphoma cells express GFP.

Cell surface molecule expression of EG7-ova hybridoma system. DC maturation express high levels of co-stimulatory molecules and immunostimulatory cytokines, which indicates that DCs are phenotypically and functionally mature state. To determine if the hybridoma expresses the dendritic cell marker and co-stimulatory molecules, 1×10⁶ hybridoma cells were collected and labeled with fluorescence antibodies against CD11c, CD40, CD80, CD86, MHC I, MHC II and MHC I-SIINFEKL. The stained samples were analyzed using flow cytometry. Both immature and mature hybridoma cell lines showed stable and similar levels of CD11c, while mature hybridoma showed an increased and stable fluorescence of CD40, CD80 and CD86. MHC I and MHC II levels for both cell lines were also observed and higher fluorescence was observed in the mature hybridoma (mBMDC-EG7-ova-GFP) cell line.

Stability of EG7-ova hybridoma system after every 10 passages. Immature and mature hybridoma cell lines were analyzed for cell surface molecule expression using fluorescence labeled antibodies against CD11c, CD40, CD80, CD86, MHC I, MHC II, and MHC I-SIINFEKL after every 10 cell passages. Immature and mature hybridoma cell lines both expressed stable and similar levels of CD11c while mature hybridoma showed an increased and stable fluorescence of CD40, CD80 and CD86. MHC I and MHC II levels for both cell lines were also observed and higher fluorescence was observed in the mature hybridoma cell line. EG7-ova cells express ovalbumin although the amount of SIINFEKL present in the EG7-ova cells have not been determined, both the immature and mature hybridoma cell line shows some expression of the MHC I-SIINFEKL. The results show that the hybridoma cell lines prove to be stable under culture conditions without losing their dendritic cell function.

Stability of EG7-ova hybridoma system after cryopreservation. Immature and mature hybridoma cells were cryopreserved in 90% FBS and cells were thawed and grown in the hybrid media. The positive clones were analyzed using a dendritic cell marker CD11c and GFP using flow cytometry. Both immature and mature hybridoma cell lines showed positive clones of about 58.8% and 75.9% approximately.

Chemically induced blebbing of hybridoma cells. Cell-free vaccines that maintain the bioactive properties of cells with enhanced scalability and storage can overcome the limitation related to cell-based vaccines. Both immature and mature hybridomas were chemically induced using N-ethylmaleimide (NEM) in 1×DPBS for 8 h at 37° C. The supernatant containing the blebs was collected followed by cell and debris removal by centrifugation at 300×g for 10 min, with subsequent micro-scale EB isolation by centrifugation at 16,100×g for 10 min. EBs were rinsed three times with 1×DPBS to remove any elements of the chemical blebbing buffer. The blebs were analyzed for its cell surface molecule expression by analyzing the bleb using flow cytometry. The blebs were labeled with fluorescence antibodies against CD11c, CD40, CD80, CD86, MHC I, MHC II, and MHC I-SIINFEKL and analyzed using flow cytometry. Both immature EB and mature EB hybridomas expressed a similar level of CD11c, a DC marker, and their co-stimulatory marker expression levels were corresponding to those of the parent hybridoma cells.

Preparation and characterization of EBs derived from the hybridoma of BMDC and T lymphoma cell. The stock solution of 2 mM N-ethylmaleimide (NEM, ThermoFisher) was prepared by dissolving 0.275 g in 10 mL DI water at a concentration and filtering the solution using a sterile 0.22 μm syringe filter. Blebbing buffer containing 0.22 mM NEM was prepared by diluting 90 μL NEM stocking solution to 10 mL DPBS (ThermoFisher) immediately before use. For blebbing, the hybridomas of BMDC and T lymphoma cell were incubated in the blebbing buffer for 6 h at 37° C. and 5% C02 to produce micro-sized EBs. The supernatant was then centrifuged at 1,000×g for 5 min to pellet un-blebbed cells and cell debris and the remaining supernatant was centrifuged at 16,100×g for 15 min. Collected EBs were further rinsed 3 times with DPBS to remove any residual blebbing reagents via repeated centrifugation at 16,100×g for 10 min. The resulting EBs were finally resuspended in DPBS and confirmed to be free of cells or cell debris under a microscope. Bicinchoninic acid (BCA) assay using a Pierce BCA protein assay kit (ThermoFisher) was used to determine the equivalent protein amounts in the cells, hybridomas, and EBs using bovine serum albumin (BSA) as a standard. Briefly, the cells, hybridomas, and EBs were lysed with 1×RIPA buffer (Cell Signaling Technology, Danvers, MA) and 25 μL of the samples were combined with BCA working reagent (200 μL). The samples were incubated at 37° C. for 30 min in a 96-well plate before reading at 562 nm using a SpectraMax Plus plate reader (Molecular Devices, San Jose, CA). BCA working reagent was prepared by mixing at a 50:1 volume ratio of BCA stock solution to a sample, following the manufacturer's specifications.

Antigen presentation to CD8 T cells by EBs derived from the hybridoma of BMDC and T lymphoma cell. The hybridomas of BMDC and T lymphoma cell, serially diluted from 3×10⁴ per well of a 96 well plate, or EBs at an equivalent surface area were incubated with 9×10⁴ CD8 B3Z hybridoma cells in RPMI hybrid media for 24 h. After centrifugation at 300×g for 10 min, the supernatant was carefully removed by a pipettor and 100 μL per well of a buffer consisting of 10% (v/v) NP-40 (Sigma Aldrich) and 0.6 mg/mL chlorophenol red-β-D-galactopyranoside (CPRG, Sigma Aldrich) in DPBS was added at, followed by an incubation at 37° C. and 5% C02 up to 3 h. The relative T cell activation was measured by the absorbance at 585 nm of catalyzed CPRG by β-galactosidase using a BioTEK Synergy HIplate reader and compared with that from B3Z cells incubated with 3×10⁴ BMDCs at varying SIINFEKL (OVA257-264, Invivogen, San Diego, CA) concentrations ranging from 0-0.1 mg/mL in 100 μL media.

Vaccination by EBs derived from the hybridoma of BMDC and T lymphoma cell. Mice were subcutaneously injected in the right flank with 50 μL of DPBS, 50 μM OVA protein, 2.5×10⁴ immature or mature BMDCs, or EBs derived from the hybridoma of BMDC and T lymphoma cell at an equivalent protein amount, twice in two weeks (prime and booster injections). In 10 days from the booster injection, 1×10⁶ E.G7-OVA or EL4 cells, which were GFP-free to avoid its antigenic properties, were subcutaneously injected in the right flank of the vaccinated mice, and tumor size was measured every 2 days using a digital caliper and calculated by V=0.5 LW², where L and W represent the longest length in one direction and the shortest width in another direction, respectively. Mice were sacrificed by C02 asphyxiation and cervical dislocation when tumors surpassed 15 mm in any direction of measurement, according to the animal use protocols. A total of 6 mice per treatment group were used for statistical significance with a power of 0.9 with an alpha equaled to 0.05. A minimum increase of 10% was considered meaningful for ultimate survival in comparison to the control and p value lower than 0.05 was considered statistically significant.

In order to assess the activation of cytotoxic T lymphocytes (taCTLs) upon vaccination, mice vaccinated as previously described were sacrificed 10 days after the booster vaccination, and the spleens were harvested. Splenocytes were obtained by maceration through a 40 m sterile tissue strainer in 5 cm² cell culture dishes in 5 mL splenocyte media (RPMI media, containing 10% FBS, 1% penicillin-streptomycin, and 0.1% β-mercaptoethanol), centrifugation at 350×g for 10 min, and RBC depletion using 1 mL of cold 1×RBC lysis buffer, resulting in approximately 1×10⁸ viable splenocytes per spleen as checked by trypan blue exclusion assay. E.G7-OVA cells labeled with CellTrace Blue (ThermoFisher) as the manufacturer recommended were incubated with the splenocytes at an E:T (splenocyte:E.G7-OVA) ratio of 25:1 for 4 h in a round bottom 96 well plate (ThermoFisher). After centrifugation at 300×g for 10 min, the cells were rinsed once with PBS and incubated with 1 μL/mL Yo-Pro-1 (ThermoFisher) for 15 min on ice. After additional rinsing three times with PBS, the specific lysis was determined as a ratio of the double-positive cells for CellTrace Blue and Yo-Pro-1 to those positive for CellTrace Blue only using flow cytometry (BD Fortessa flow cytometer, BD Biosciences, Franklin Lakes, NJ).

Statistical analysis. For all in vitro studies, triplicate data were analyzed and presented as average±standard deviation. To achieve statistical significance in in vivo studies, 6 animals per treatment group (n=6) were used. Two-tailed Student's t-test (GraphPad Prism Ver. 8.0.1) was used to calculate the statistical significance of comparisons between two groups, and p-values less than 0.05 were considered significant. Kaplan-Meier curves and Mantel-Cox tests were used to analyze survivals between groups.

T cell activation ability of hybridoma cell. Immature and mature hybridoma cell line and their respective EBs were tested for T cell stimulation ability in vitro using a SIINFEKL presentation responsive T cell hybridoma. When dosed by equivalent surface area, EBs demonstrated similar T cell stimulation compared to the parent cells, with mature hybridomas and EBs stimulating T cells 2 times more than the immature phenotypes.

Vaccination of EG7-ova hybridoma blebs for protection from OVA-expressing tumor. C57BL/6 mice were immunized twice in 14 days (prime and boost), and 10 days after booster shot 1×10⁶ E.G7-OVA cells were subcutaneously injected into the right-hand flank. Body weight, tumor volume, and survival were recorded. Overall, the body weight did not decrease. The tumor volume was measured every three days. PBS and OVA vaccines were not effective at preventing tumor growth, with all mice demonstrating tumor growth and death within 18 and 27 days, respectively. As for the immature BMDC and immature hybridoma EBs, there was delay in tumor growth while the mature BMDCs and mature EB hybridoma showed consistent tumor growth.

Splenocyte specific lysis of EG7-ova hybridoma. C57BL/6 mice were immunized twice in 14 days (prime and boost), and 10 days after booster shot, spleens were collected and analyzed for specific lysis. The splenocytes isolated from the mice were incubated with E.G7-OVA T cell lymphoma for 4 hours followed by analysis using flow cytometry. Cancer cells were labeled with cell trace blue and the analyzed against the percentage of YO-PRO-1 positive cells by flow cytometry. At an E:T (effector:target) ratio of 50:1, ˜27% of E.G7-OVA cells were specifically killed by the OVA-vaccinated mice, ˜65% in iBMDC vaccinated mice, ˜80% in mBMDC mice, ˜68% in immature hybridoma EB vaccinated mice and ˜90% mature hybridoma EB. Based on these results, hybridoma EBs are efficient for T cell activation.

EL4 T lymphoma cell fusion with bone marrow dendritic cells. To compare the effect of EG7-ova hybridomas, EL4 cells were used as a control cell to determine the function of hybridoma blebs. BMDCs were obtained by incubating the bone marrow cells from C57BL/6 mice femurs, with 20 ng/mL GM-CSF for 5 days. The resulting BMDCs (˜80% CD11c+) were then incubated with or without 20 ng/mL LPS for 24 h, to prepare mBMDCs and immature DCs (iBMDCs), respectively. The BMDCs were fused with EL4 T lymphoma cells using polyethylene glycol (PEG) at a 5:1 ratio in serum free media. The fused cells were incubated for 15 minutes at 37° C. PEG was washed using serum free media at 300×g for 10 minutes for a total of two washes. The fused cells were placed in RPMI media containing 10% FBS, 1% pen-strep in presence of GM-CSF and cultured for 6 days prior to single cell sorting using CD11c (dendritic cell marker) and RFP by flow cytometry. The DC-T cell hybridomas were maintained for growth in RPMI with 10% FBS and 1% pen-strep. The resulting hybridoma showed a positive clone for CD11c, a representative DC marker

EL4 hybridoma stability. Immature and mature hybridoma cell lines were analyzed for cell surface molecule expression using fluorescence labeled antibodies against CD11c, CD40, CD80, CD86, MHC I, and MHC II. Immature and mature hybridoma cell lines both expressed stable and similar levels of CD11c while mature hybridoma showed an increased and stable fluorescence of CD40, CD80, and CD86. MHC I and MHC II levels for both cell lines were also observed and higher fluorescence was observed in the mature hybridoma cell line.

Vaccination of EL4 hybridoma blebs for anti-tumor protection. C57BL/6 mice were immunized twice in 14 days (prime and boost), and 10 days after booster shot 1×10⁶ EL4 T lymphoma cells were subcutaneously injected into the right-hand flank. Body weight, tumor volume, and survival were recorded. Overall, the body weight did not decrease. The tumor volume was measured every three days and PBS and iBMDC vaccines were not effective at preventing tumor growth, with all mice demonstrating tumor growth and death within 12 and 15 days, respectively. As for the immature BMDC and immature hybridoma EBs, there was delay in tumor growth while the mature BMDCs and mature hybridoma EBs shows consistent tumor growth.

Splenocyte specific lysis of EL4 hybridoma blebs. C57BL/6 mice were immunized twice in 14 days (prime and boost), and 10 days after booster shot, spleens were collected and analyzed for specific lysis. The splenocytes isolated from the mice were incubated with EL4 T cell lymphoma for 4 hours followed by analysis using flow cytometry. Cancer cells were labeled with cell trace blue and the analyzed against the percentage of YO-PRO-1 positive cells by flow cytometry. At an E:T (effector:target) ratio of 50:1, approximately 55% and 30% of EL4 and E.G7-OVA cancer cells respectively were lysed by splenocytes in the mature hybridoma EB groups and showed the highest specific lysis as compared to the other groups. Both immature BMDCs and EBs showed similar specific lysis to cancer cells. From previous data using E.G7-OVA hybridoma vaccine system, the hybridoma vaccine system shows to be antigen specific in terms of lysing specific cancer cells.

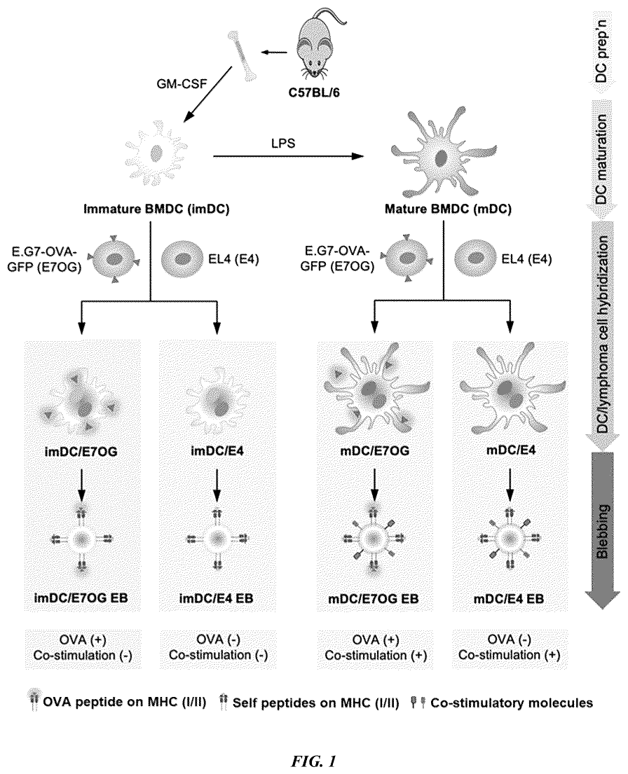

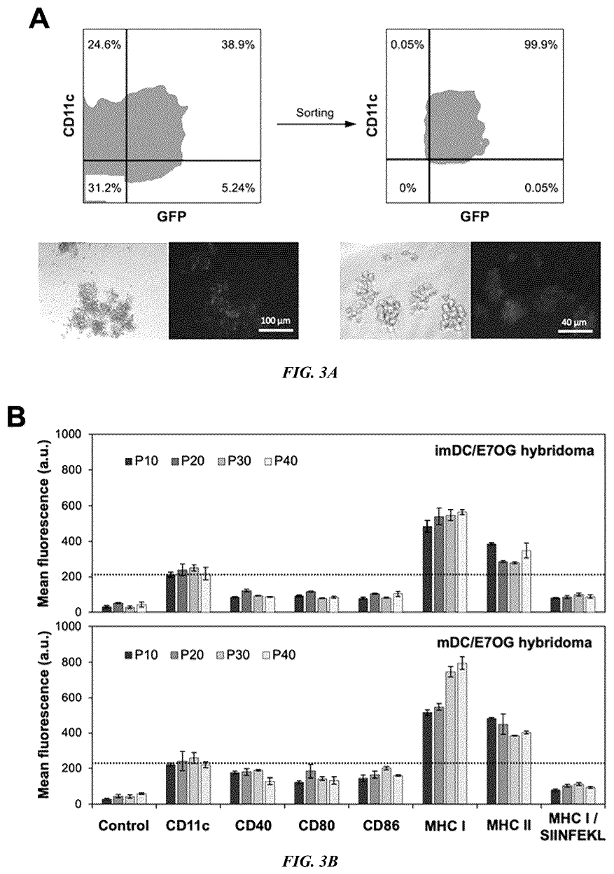

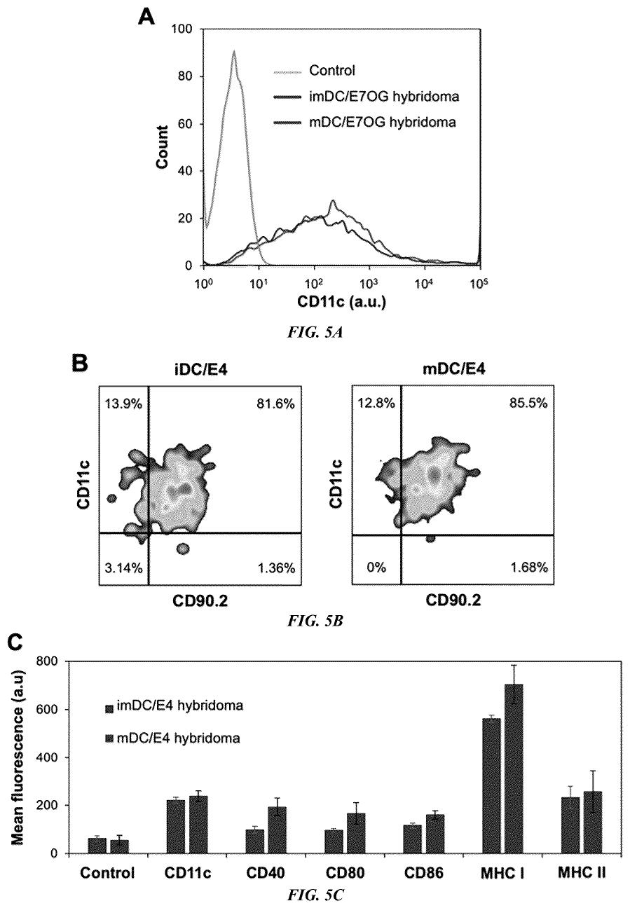

Hybridomas with stably preserved functions of BMDC and T lymphoma cell. One of the pivotal questions in developing cell-based vaccines is the longevity of the immunological functions such as sustained antigen presentation and co-stimulation. This study fused BMDCs at a controlled maturation with T lymphoma cells with or without a known antigen (see FIG. 1). Briefly, the bone marrow harvested from C57BL/6 mice were differentiated to BMDCs in the presence of GM-CSF with or without further maturation by incubation with lipopolysaccharide (LPS), preparing immature and mature BMDCs (imDCs and mDCs). T lymphoma cells derived from C57BL/6 with or without further modification for the expression of ovalbumin (OVA), E.G7-OVA and EL4 cells, were then hybridized with an imDC or a mDC via a simple polyethylene glycol (PEG)-mediated cell fusion. For straightforward selection, E.G7-OVA was transfected by retroviral vectors for GFP expression, resulting in E.G7-OVA-GFP (E7OG), while no modification was done to EL4 as a model tumor with no known antigens (E4 cells). The four different hybridomas, imDC/E7OG, imDC/E4, mDC/E7OG, and mDC/E4, were analyzed for double stained CD11c and GFP as markers for a DC and E7OG cell, respectively, by flow cytometry (see FIGS. 3A and 5A). The hybridization of a BMDC and E4 cell was confirmed by double-staining for CD11c and CD90.2. Hybridoma were noticeable in a day as cell clumps (see FIG. 3A). Pure mDC/E7OG hybridoma was obtained after sorting in the live cell population identified by staining with propidium iodide (PI) and co-expressing CD11c and GFP, followed by being grown to confluency. The sorting increased the hybridoma's population from approximately 40% to nearly 100%, as confirmed by flow cytometry and uniform GFP expression (see FIGS. 3A and 5B). Hybridomas prepared via successful cell fusion have been shown for their extended biological stability. The sustained capability of the DC/T lymphoma cell hybridomas for antigen presentation and co-stimulation was assessed by analyzing them for CD11c, CD40, CD80, CD86, MHC I (including those presenting OVA-derived SIINFEKL), and MHC II on every 10 passages (see FIG. 3B). While CD11c expression was consistently maintained in all passages regardless of the maturation status of the parent DCs, co-stimulatory and maturation markers, including CD40, CD80, and CD86 as well as MHC II, were higher in the hybridomas derived from mDCs than in those from iDCs, indicating the preserved immunological functions of the parent DCs in the corresponding hybridomas. DC/EL4 hybridomas also showed the preserved maturation status of the parent DC (see FIG. 5C). The results in FIG. 3 and FIG. 5, demonstrated the successful generation of the hybridomas with varying DC maturation statuses and antigenic profiles before preparing cell-free, cell-mimicking extracellular bleb (EB) vaccines.

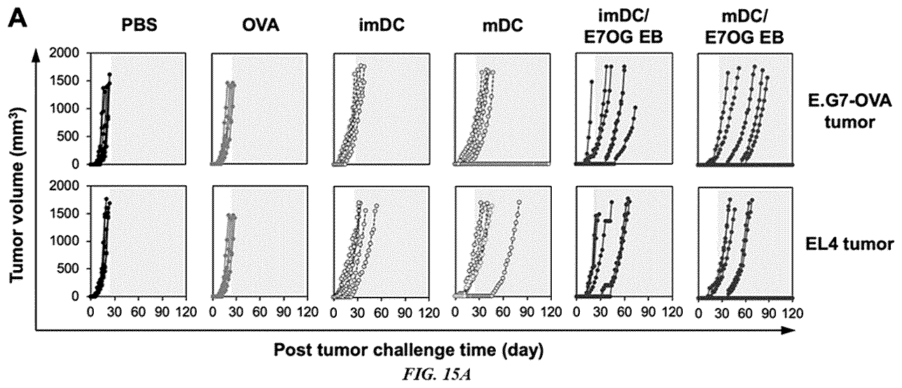

Preserved antigen presentation and co-stimulation by the hybridoma and their EBs. As major antigen presenting cells (APCs), DCs are capable of presenting antigens to CD4+ and CD8+ T cells along with co-stimulatory signals in eliciting highly orchestrated cellular and humoral immune responses, making them a promising cell-based immunotherapeutic, such as vaccines against cancer. Despite the proven immunogenicity and favorable safety profiles from clinical studies using autologous and allogenic DCs, DC-based immunotherapies have not succeeded in warranting measurable clinical benefits yet. Shared by many forms of cell therapies such as CAR T cells, DCs are also known to adapt to suppressive microenvironment and lose their immune-activating functions. In addition, tumor antigens must be taken up and processed by DCs for desirable antigen processing and presentation, which multi-dimensional hurdles. In attempt to overcoming the DC-based vaccines by creating cell-free, DC-mimicking vaccines, the DC/T lymphoma hybridomas were converted to micro-sized EBs via chemically-induced blebbing which generate membrane vesicles including soluble cytosolic contents without intracellular organelles and cytoskeletons (see FIG. 12A). The analyses of the surface markers on the hybridomas and corresponding EBs, including CD11c, CD40, CD80, CD86, MHC I (including those presenting SIINFEKL), and MHC II confirmed exact preservation during the chemically induced blebbing, depending on the maturation statuses of the parent DCs (see FIG. 12B). The EBs' capability to present antigens and activate T cells were measured by incubating them with CD8 T cell hybridoma, B3Z cells. B3Z cells are engineered to secret LacZ upon receiving SIINFEKL presented on MHC (H-2K^{b}) of APCs. It was shown that the DC's antigen presentation capabilities of activating T cells were identically translated to the corresponding EBs (see FIG. 12C) in a DC's maturation status-dependent manner. The results clearly demonstrated that the acellular EBs closely mimic not only surface molecules but also interactions with T cells, rendering them a promising alternative to DC-based vaccines.

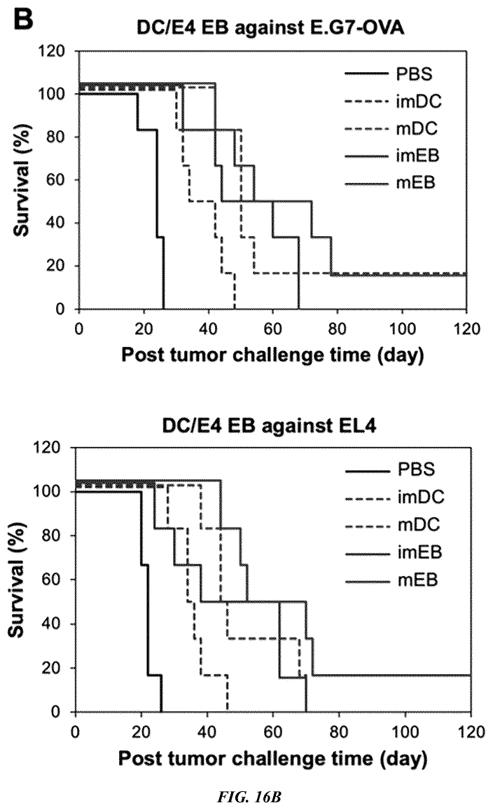

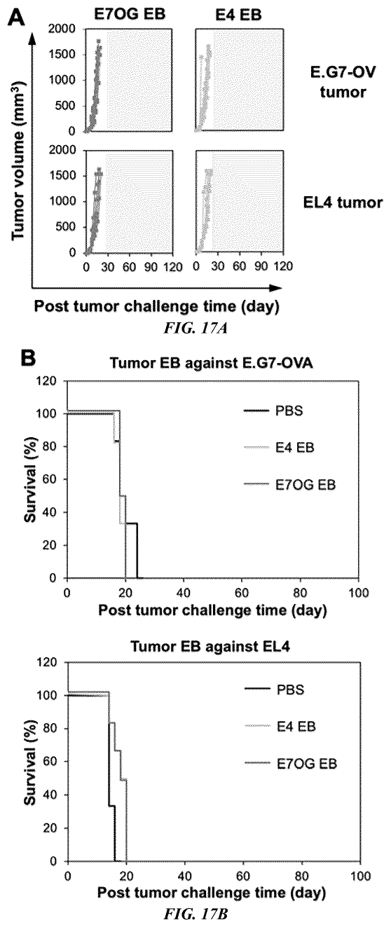

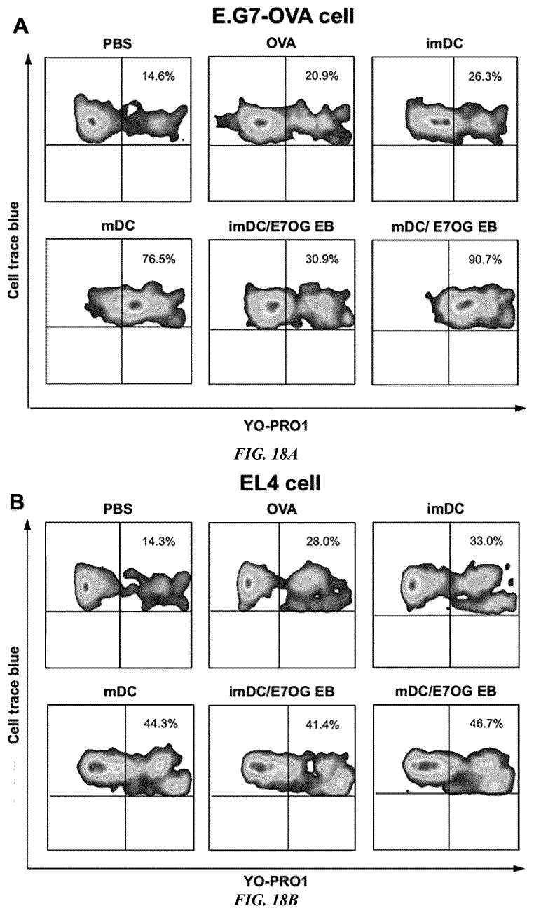

Protection of tumor-challenged animals by EB vaccines. Based on the in vitro confirmation of the preserved antigen presentation and T activation by the EBs derived from the corresponding hybridomas of DC/T lymphoma cell, C57BL/6 mice were vaccinated twice 14 days apart with PBS, OVA (protein vaccine), imDCs or mDCs (antigen-free cell-based vaccines), or EBs derived from varying hybridomas of DC/T lymphoma cell via subcutaneous injection based on equivalent protein concentration prior to challenging the animals with E.G7-OVA (E7OG, free of GFP to avoid its immunogenicity) or EL4 (E4) cells 10 days after the second (booster) injection. While the tumors in the mice vaccinated with PBS or OVA resulted in rapid progression, vaccination by DCs or EBs efficiently suppressed the tumor growth and extended the survival of the animals (see FIGS. 15 and 16; and Tables 1-4). Regardless of the vaccines and challenged tumor types, the maturation status of mDCs and mDC/T lymphoma cell-derived EBs were more efficient than immature counterparts. In addition, EBs were more efficient than the corresponding DCs in protecting the animals, possible due to their extended stability without being adaption to immune suppressive environment unlike DCs. As expected, mDC/E7OG EBs were the most efficient against E.G7-OVA tumor due to their costimulatory capability against a tumor with an antigen (see FIG. 15). More notably, mDC/E4 EBs were also effective against EL4 tumor (see FIG. 16). This implies that presenting the exact molecular profile of cancer cells could be specific for the immune system to recognize the target in the absence of a distinct antigen. However, EBs derived from E.G7-OVA and EL4 cells were not able to affect the tumor growth (see FIG. 17), indicating that it is critical to provide a molecular profile of cancer cells along with appropriate immunological signals, which was accomplished by merging a cancer cell with a mDC. The results demonstrate the efficient protection of tumor-challenged animals by the EBs derived from the hybridomas of mDC/T lymphoma cell.

TABLE 1
DC/E7OG EB against E.G7-OVA tumor

| | PBS | OVA | imDC | mDC | iEB |
|---|---|---|---|---|---|
| OVA | NS 0.2157 | | | | |
| imDC | ** 0.0015 | ** 0.0044 | | | |
| mDC | ** 0.0037 | ** 0.0013 | NS 0.0719 | | |
| iEB | * 0.0144 | * 0.0338 | NS 0.7503 | NS 0.1233 | |
| mEB | ** 0.0015 | ** 0.0013 | *0.0484 | NS 0.3367 | NS 0.9367 |

TABLE 2
DC/E7OG EB against EL4 tumor

| | PBS | OVA | imDC | mDC | iEB |
|---|---|---|---|---|---|
| OVA | ** 0.0045 | | | | |
| imDC | ** 0.0016 | ** 0.0013 | | | |
| mDC | ** 0.0016 | ** 0.0013 | NS 0.1217 | | |
| iEB | ** 0.0016 | ** 0.0021 | NS 0.7495 | NS 0.3367 | |
| mEB | ** 0.0016 | ** 0.0013 | NS 0.1217 | NS 0.9365 | NS 0.3382 |

TABLE 3
DC/E4 EB against E.G7-OVA tumor

| | PBS | OVA | imDC | mDC | iEB |
|---|---|---|---|---|---|
| imDC | * 0.0144 | NS 0.0552 | | | |
| mDC | ** 0.0015 | ** 0.0013 | NS 0.2283 | | |
| iEB | ** 0.0015 | ** 0.0013 | NS 0.3870 | NS 0.8092 | |
| mEB | ** 0.0015 | ** 0.0013 | NS 0.0872 | NS 0.6325 | NS 0.5523 |

TABLE 4
DC/E4 EB against EL4 tumor

| | PBS | OVA | imDC | mDC | iEB |
|---|---|---|---|---|---|
| imDC | ** 0.0016 | ** 0.0013 | | | |
| mDC | ** 0.0016 | ** 0.0013 | NS 0.2964 | | |
| iEB | ** 0.0016 | ** 0.0021 | NS 0.8116 | NS 0.2268 | |
| mEB | ** 0.0016 | ** 0.0013 | NS 0.1045 | NS 0.6900 | NS 0.1045 |

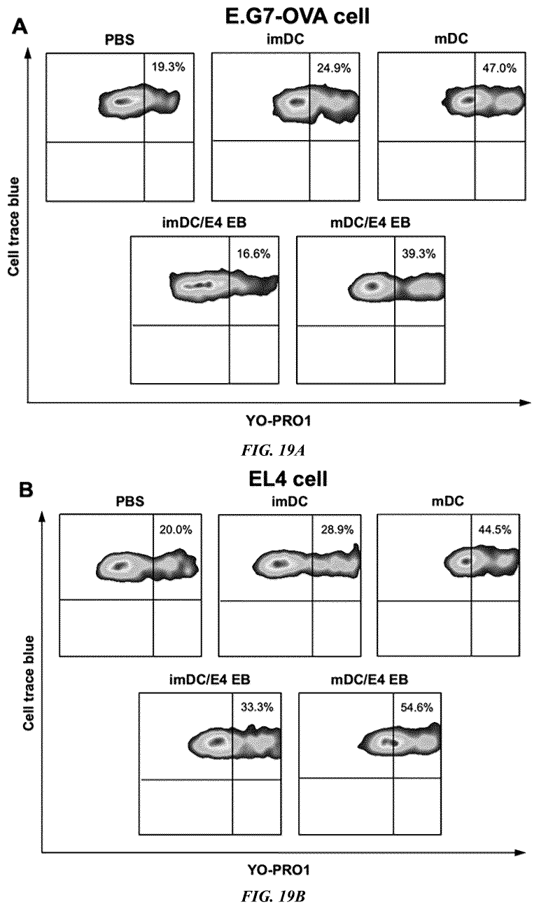

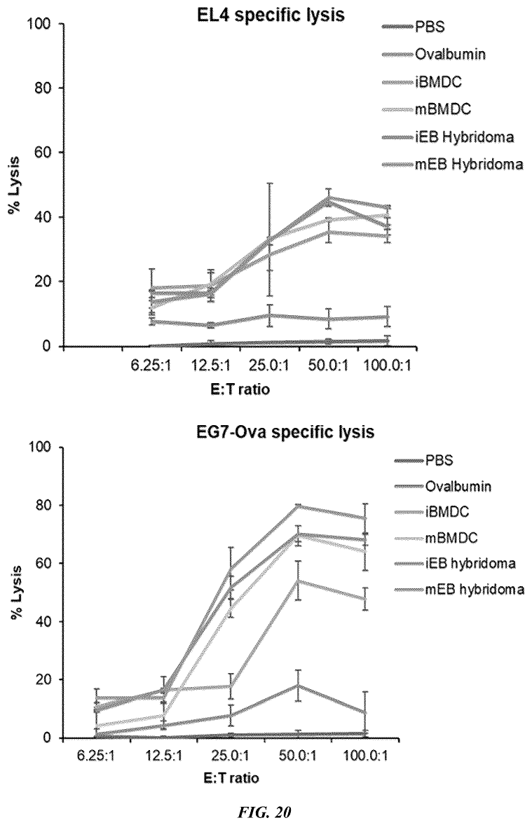

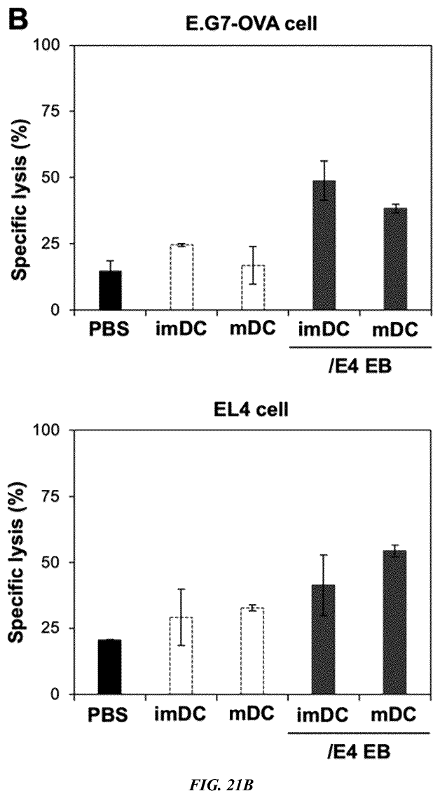

Elicited cytotoxic T lymphocyte (CTL)-mediated response by EBs. In general, CTLs play efficient roles in eradicating cancer cells, requiring antigen presentation by MHC I for direct CD8 T cell activation and MHC II for T helper cell generation. To confirm whether the effective protection of the tumor-challenged mice was attributed to CTL activation by the EBs derived from the hybridoma of DC/T lymphoma cell, C57BL/6 mice were vaccinated as in the tumor-challenge study, followed by harvesting splenocytes 10 days after the booster injection. The splenocytes that include activated T cells were incubated with E.G7-OVA or EL4 cells, followed by quantifying specific lysis of the cancer cells by flow cytometry as previously reported. At an effector/splenocyte to tumor cell ratio (E:T) of 25, approximately 92% of E.G7-OVA cells (see FIGS. 18A and 21A) and 49% of EL4 (see FIGS. 18B and 21B) cells were specifically lysed by the splenocytes harvested from the mice vaccinated with mDC/E7OG EBs. As anticipated from the tumor-challenge study (see FIG. 16), the splenocytes harvested from the mice vaccinated with mDC/E4 EBs (i.e., no target antigen), demonstrated approximately 38% and 55% specific lyses of E.G7-OVA and EL4 cells, respectively (see FIG. 19 and FIG. 21). From these findings, it is clear that vaccination against a known antigen warrants the most efficient CTL activation against the cancer with the corresponding antigen as similarly observed in FIG. 15. Notably, vaccination against no distinct target was still effective in CTL activation against the matching tumor, consistent with the tumor-challenging study (see FIG. 16). The fact that the specific lysis of E.G7-OVA cells by the splenocytes harvested from the mice vaccinated with mDC/E4 EBs was lower than that of EL4 cells may implicate the more important roles of targeting the full molecular profile than a single distinct antigen in activating anti-tumor immune response. The results shown in FIGS. 15, 16, 18 and 19 cohesively demonstrated the efficacy of DC/T lymphoma EB vaccines against the corresponding tumor, contributed by specific CTL activation.

It will be understood that various modifications may be made without departing from the spirit and scope of this disclosure. Accordingly, other embodiments are within the scope of the following claims.

1. A vaccine preparation comprising extracellular blebs from a hybridoma of an antigen presenting cell and a target cell, wherein the hybridoma expresses an antigen that can modulate a subject's immune system,
wherein the extracellular blebs are produced from the hybridoma by treating the hybridoma with a blebbing agent, and
wherein the antigen is displayed on the surface of the extracellular blebs.

2. The vaccine preparation of claim 1, wherein the antigen presenting cell is selected from a macrophage, a B cell, and a dendritic cell.

3. The vaccine preparation of claim 2, wherein the dendritic cell is a mature dendritic cell or an immature dendritic cell.

4. (canceled)

5. The vaccine preparation of claim 2, wherein the dendritic cell is a bone marrow derived dendritic cell, a monocyte-derived dendritic cell, or a peripheral blood mononuclear cell-derived dendritic cell.

6. (canceled)

7. The vaccine preparation of claim 1, wherein the target cell is selected from a cancer cell, an abnormal or diseased cell, a cell engineered to display an antigen, or a cell that is infected with an infectious agent.

8. The vaccine preparation of claim 7, wherein the target cell is a cancer cell selected from a myeloma, a lymphoma, a carcinoma, a sarcoma, a leukemia, an adenocarcinoma, a thymoma, or a neoplastic cell from a malignant tumor.

9. The vaccine preparation of claim 7, wherein the target cell is a cell that is infected by a virus, a fungus, or a bacterium.

10. The vaccine preparation of claim 9, wherein the virus is selected from Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Coronavirus, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, Epstein-Barr virus, European bat lyssavirusalitis, GB virus C/Hepatitis G virus Pegivirus, Hantan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus, Human herpesvirus, Human immunodeficiency virus, Human papillomavirus, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Isfahan virus, JC polyomavirus, Japanese encephalitis virus, Junin arenavirus, KI Polyomavirus, Kunjin virus, Lagos bat virus, Lake Victoria Marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, Semliki forest virus, Seoul virus, Simian foamy virus, Simian virus, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus O, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, and Zika virus.

11. The vaccine preparation of claim 9, wherein the fungus is selected from Absidia corymbifera, Absidia ramose, Achorion gallinae, Actinomadura spp., Ajellomyces dermatididis, Aleurisma brasiliensis, Allersheria boydii, Arthroderma spp., Aspergillus flavus, Aspergillus fumigatu, Basidiobolus spp., Blastomyces spp., Cadophora spp., Candida albicans, Cercospora apii, Chrysosporium spp., Cladosporium spp., Cladothrix asteroids, Coccidioides immitis, Cryptococcus albidus, Cryptococcus gattii, Cryptococcus laurentii, Cryptococcus neoformans, Cunninghamella elegans, Dematium wernecke, Discomyces israelii, Emmonsia spp., Emmonsiella capsulate, Endomyces geotrichum, Entomophthora coronate, Epidermophyton floccosum, Filobasidiella neoformans, Fonsecaea spp., Geotrichum candidum, Glenospora khartoumensis, Gymnoascus gypseus, Haplosporangium parvum, Histoplasma, Histoplasma capsulatum, Hormiscium dermatididis, Hormodendrum spp., Keratinomyces spp., Langeronia soudanense, Leptosphaeria senegalensis, Lichtheimia corymbifera, Lobmyces loboi., Loboa loboi, Lobomycosis, Madurella spp., Malassezia furfur, Micrococcus pelletieri, Microsporum spp., Monilia spp., Mucor spp., Mycobacterium tuberculosis, Nannizzia spp., Neotestudina rosatii, Nocardia spp., Oidium albicans, Oospora lactis, Paracoccidioides brasiliensis, Petriellidium boydii, Phialophora spp., Piedraia hortae, Pityrosporum furfur, Pneumocystis jirovecii (or Pneumocystis carinii), Pullularia gougerotii, Pyrenochaeta romeroi, Rhinosporidium seeberi, Sabouraudites (Microsporum), Sartorya fumigate, Sepedonium, Sporotrichum spp., Stachybotrys, Stachybotrys chartarum, Streptomyce spp., Tinea spp., Torula spp., Trichophyton spp., Trichosporon spp., and Zopfia rosatii.

12. The vaccine preparation of claim 9, wherein the bacterium is selected from Actinomyces israelii, Bacillus anthracis, Bacillus cereus, Bartonella henselae, Bartonella quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Francisella tularensis, Haemophilus influenzae, Helicobacter pylon, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumoniae, Neisseria gonorrhoeae, Neisseria meningitidis, Pseudomonas aeruginosa, Rickettsia rickettsia, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Ureaplasma urealyticum, Vibrio cholerae, Yersinia pestis, Yersinia enterocolitica, and Yersinia pseudotuberculosis.

13. The vaccine preparation of claim 1, wherein the antigen presenting cell and/or target cell is from a subject that is to be treated with the vaccine preparation.

14. The vaccine preparation of claim 1, wherein the antigen comprises a tumor-specific antigen, or a tumor-associated antigen.

15. (canceled)

16. The vaccine preparation of claim 1, wherein the antigen is a foreign antigen or a self-antigen.

17. (canceled)

18. (canceled)

19. The vaccine preparation of claim 1, wherein the vaccine preparation further comprises an adjuvant.

20. (canceled)

21. The vaccine preparation of claim 1, wherein the extracellular blebs comprise one or more of the following surface and maturation markers CD11c, MHC I, CD40, CD80, and/or CD86.

22. A method of making the vaccine preparation of claim 1, comprising:
generating extracellular blebs from a hybridoma by contacting the hybridoma with the one or more sulfhydryl blocking agents for 30 min to 24 h;
isolating the extracellular blebs.

23. (canceled)

24. The method of claim 22, wherein the one or more sulfhydryl blocking agents is N-ethylmaleimide or paraformaldehyde.

25. (canceled)

26. A method of immunizing a subject in need thereof, comprising administering a therapeutically effective amount of the vaccine preparation of claim 1 to the subject.

27. The method of claim 26, wherein the vaccine preparation is administered intramuscularly, subcutaneously, intradermally, or intratumorally.

28. The method of claim 26, wherein the subject has cancer, an autoimmune disease, a neurodegenerative disorder, or an infection by a pathogen.
